SEQUENCE LISTING (1 sequence)

SEQ ID NO: 1 - protein, 540 aa, Artificial Sequence
  Composite GII.4 Norovirus VP1 amino acid sequence
MKMASSDANPSDGSTANLVPEVNNEVMALEPVVGAAIAAPVAGQQNVIDPWIRNNFVQAPGGEFTVSPRNAPGEILWSAPLGPDLNPYLSHLARMYNGYAGGFEVQVILAGNAFTAGKIIFAAVPPNFPTEGLSPSQVTMFPHIIVDVRQLEPVLIPLPDVRNNFYHYNQSNDPTIKLIAMLYTPLRANNAGDDVFTVSCRVLTRPSPDFDFIFLVPPTVESRTKPFTVPILTVEEMTNSRFPIPLEKLFTGPSGAFVVQPQNGRCTTDGVLLGTTQLSPVNICTFRGDVTHIAGTQEYTMNLASQNWNNYDPTEEIPAPLGTPDFVGKIQGVLTQTTRGDGSTRGHKATVSTGSVHFTPKLGSVQFSTDTSNDFETGQNTKFTPVGVVQDGSTTHQNEPQQWVLPDYSGRDSHNVHLAPAVAPTFPGEQLLFFRSTMPGCSGYPNMNLDCLLPQEWVQHFYQEAAPAQSDVALLRFVNPDTGRVLFECKLHKSGYVTVAHTGQHDLVIPPNGYFRFDSWVNQFYTLAPMGNGTGRRRAL

(19) United States
(12) Patent Application Publication
(10) Pub. No.: US 2022/0054621 A1
(43) Pub. Date: Feb. 24, 2022

(54) NOROVIRUS VACCINE FORMULATIONS AND METHODS

(71) Applicant: TAKEDA VACCINES, INC., CAMBRIDGE, MA (US)

(72) Inventors: Taisei MASUDA, Tokyo (JP); James SHERWOOD, Cambridge, MA (US); Paul MENDELMAN, Cambridge, MA (US); Frank BAEHNER, Konstanz (DE)

(21) Appl. No.: 17/416,810

(22) PCT Filed: Dec. 20, 2019
(86) PCT No.: PCT/US2019/067961

§ 371 Date: Jun. 21, 2021

Related U.S. Application Data
(60) Provisional application No. 62/782,733, filed on Dec. 20, 2018.

(51) Int. Cl. A61K 39/12; A61P 31/14

(52) U.S. Cl. CPC A61K 39/12; A61K 2039/5258; A61P 31/14

(57) ABSTRACT

The invention is in the field of vaccines, particularly vaccines for Noroviruses. In addition, the invention relates to methods of preparing vaccine compositions and methods of inducing and evaluating protective immune responses against Norovirus in humans, in particular, pediatric patients.

### CROSS REFERENCE TO RELATED APPLICATIONS

This application claims the benefit of Provisional Application 62/782,733, filed on Dec. 20, 2018, the content of which is herein incorporated by reference in its entirety.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: LIGO_028_01WO_SeqList_ST25, date recorded Dec. 13, 2019, file size 5 kilobytes).

### BACKGROUND OF THE INVENTION

Noroviruses are non-cultivatable human Caliciviruses that have emerged as the single most important cause of epidemic outbreaks of nonbacterial gastroenteritis (Glass et al., 2000; Hardy et al., 1999). The clinical significance of Noroviruses was under-appreciated prior to the development of sensitive molecular diagnostic assays. The cloning of the prototype genogroup I Norwalk virus (NV) genome and the production of virus-like particles (VLPs) from a recombinant Baculovirus expression system led to the development of assays that revealed widespread Norovirus infections (Jiang et al. 1990; 1992).

Noroviruses are single-stranded, positive sense RNA viruses that contain a non-segmented RNA genome. The viral genome encodes three open reading frames, of which the latter two specify the production of the major capsid protein and a minor structural protein, respectively (Glass et al. 2000). When expressed at high levels in eukaryotic expression systems, the capsid protein of NV, and certain other Noroviruses, self-assembles into VLPs that structurally mimic native Norovirus virions. When viewed by transmission electron microscopy, the VLPs are morphologically indistinguishable from infectious virions isolated from human stool samples.

Immune responses to Noroviruses are complex, and the correlates of protection are just now being elucidated. Human volunteer studies performed with native virus demonstrated that mucosally-derived memory immune responses provided short-term protection from infection and suggested that vaccine-mediated protection is feasible (Lindesmith et al. 2003; Parrino et al. 1977; Wyatt et al., 1974).

Protective immunity against Norovirus in humans remains elusive because the indicators of a protective immune response in humans have still not been clearly identified (Herbst-Kralovetz et al. (2010) Expert Rev. Vaccines 9(3), 299-307). There is a need in the art to identify safe, effective methods for eliciting protective immunity against Norovirus infection, in particular in vulnerable patient populations such as infants and children.

### SUMMARY OF THE INVENTION

The present disclosure provides a method of eliciting protective immunity against Norovirus in a human pediatric subject comprising administering parenterally to the subject an effective amount of a vaccine composition, said vaccine composition comprising Norovirus VLPs. In embodiments, the compositions comprise genogroup I Norovirus VLPs and genogroup II Norovirus VLPs. In embodiments, the methods comprise administering at least a first dose and a second dose of the composition to the subject. The disclosure also provides compositions comprising genogroup I Norovirus VLPs and genogroup II Norovirus VLPs for use in a method of eliciting protective immunity against norovirus in a pediatric subject. The disclosure further provides uses of compositions comprising genogroup I Norovirus VLPs and genogroup II Norovirus VLPs in methods for eliciting protective immunity against Norovirus infection in pediatric subjects.

In an aspect, the compositions useful in the methods and uses provided herein comprise Norovirus genogroup I, genotype 1 (GI.1) VLPs and Norovirus genogroup II, genotype 4 (GII.4) VLPs. In some embodiments, the GII.4 VLPs are derived from expression of a consensus sequence of circulating strains of Norovirus genogroup II, genotype 4. In certain embodiments, the GII.4 VLPs comprise a capsid protein comprising a sequence of SEQ ID NO: 1. Such VLPs are referred to herein, in some embodiments, as “GII.4c.”

In embodiments, the compositions useful in the methods and uses provided herein comprise about 15 μg to about 150 μg of each VLP type in the composition. In certain embodiments, the compositions comprise about 15 μg GI.1 VLP and about 15 μg GII.4 VLP; or about 15 μg GI.1 VLP and about 50 μg GII.4 VLP; or about 50 μg GI.1 VLP and about 50 GII.4 VLP; or about 50 μg GI.1 VLP and about 150 μg GII.4 VLP. In some embodiments, the compositions further comprise one or more adjuvants. In some embodiments, the compositions comprise a single adjuvant. In some embodiments, the compositions comprise aluminum hydroxide. In some embodiments, the compositions comprise 500 μg aluminum hydroxide. In some embodiments, the compositions are formulated for intramuscular administration. Thus, in some embodiments, the present disclosure provides methods for eliciting protective immunity in a pediatric subject, comprising intramuscular administration of a composition comprising GI.1 and GII.4 VLPs as described herein, and further comprising 500 μg aluminum hydroxide.

In embodiments, the pediatric subject is between about 6 weeks and about 9 years of age. In further embodiments, the composition is administered to the subject in two or three doses. In some embodiments, the pediatric subject is between about 6 weeks and about 6 months of age. In some embodiments, the methods and uses provided herein comprise administration of no more than three doses (e.g., one, two, or three doses) of the compositions provided herein, wherein the subject is between about 6 weeks and about 6 months of age. In further embodiments, the subject is between about 6 weeks and about 6 months, and the composition is administered in two or three doses. In further embodiments, the subject is between about 6 weeks and about 6 months, and the composition is administered in exactly three doses. In some embodiments, the subject is between about 6 weeks and about 6 months, and the composition is administered in at least three doses. In some embodiments, the pediatric subject is between about 6 months of age and about 1 year of age. In some embodiments, the pediatric subject is between about 1 year of age and about 4 years of age. In some embodiments, the pediatric subject is between about 4 years of age and about 9 years of age. In some embodiments, the methods and uses provided herein comprise administration of no more than two doses of the compositions provided herein, e.g. one dose or two doses, wherein the subject is between about 6 months of age and about 9 years of age (e.g., between about 6 months and about 1 year; between about 1 year and about 4 years; or between about 4 years and about 9 years). In some embodiments, the methods and uses provided herein comprise administration of exactly two doses of the compositions provided herein, wherein the subject is between about 6 months of age and about 9 years of age (e.g., between about 6 months and about 1 year; between about 1 year and about 4 years; or between about 4 years and about 9 years).

In some embodiments, the present disclosure provides methods and uses of the compositions provided herein, wherein the first and second doses are administered to the subject about 1, about 2, or about 3 months apart. In further embodiments, the methods comprise administration of three doses of the composition, wherein the second and third doses are administered to the subject about 1, about 2, or about 3 months apart. In some embodiments, the first dose is administered to the pediatric subject when the subject is about 3, about 4, about 5, about 6, about 7, about 8, about 9, about 10, about 11, or about 12 months of age and the second dose is administered when the subject is about 5, about 6, about 7, about 8, about 9, about 10, about 11, about 12, about 13, or about 14 months of age. For example, in some embodiments, the first dose is administered to the pediatric subject when the subject is about 5 months of age and the second dose is administered when the subject is about 7 months of age.

In some embodiments, the methods and uses provided herein elicit at least a three-fold or at least a four-fold increase in Norovirus-specific serum antibody titer, as compared to the titer in the subject prior to administration of the composition. In some embodiments, the methods and uses provided herein are associated with an acceptable safety profile. For example, in some embodiments, the compositions provided herein are safely administered to pediatric subjects. In some embodiments, the compositions provided herein are well tolerated in pediatric subjects. For example, the compositions provided herein are well tolerated even when administered to pediatric subjects at the highest doses tested (e.g., 50 μg GI.1 VLP and 150 μg GII.4 VLP) and at the highest number of doses tested (e.g., two or three doses). In some embodiments, the methods and uses provided herein are associated with a statistically significant safe adverse event profile. In some embodiments, the method has a statistically significant low adverse event occurrence and/or severity. In some embodiments, the methods and uses provided herein have a low incidence of adverse events. In some embodiments, the methods and uses provided herein have a low incidence of serious adverse events. In some embodiments, tthe method has a negligible incidence of serious adverse events. In some embodiments, the methods and uses provided herein have a lower frequency and/or severity of adverse event occurrence in comparison with comparable methods and uses employing different vaccine compositions.

In some embodiments, the methods and uses provided herein induce cross-reactivity to one or more viral strains not present in the composition. For example, in some embodiments, the methods and uses provided herein induce an immune response against one or more viral strains not present in the composition. In some embodiments, the methods and uses provided herein induce protective immunity in the subject against one or more viral strains not present in the composition. The one or more viral strain not present in the composition may be a Norovirus strain not present in the composition, and/or a Norovirus strain not represented in the composition. For example, the Norovirus strain not present in the composition may be a strain other than a GI.1 or a GI1.4 Norovirus strain; or may be a GII.4 Norovirus strain that is not one of the strains used to derive the GII.4c consensus sequence.

In some aspects, the present disclosure provides methods, uses, and compositions for use in inducing protective immunity against Norovirus infection in a pediatric subject, comprising the steps of (i) determining the subject's age, and (ii)(a) if the age is at least about 6 weeks but less than 6 months, administering to the subject a composition provided herein comprising Norovirus VLPs in a dosing regimen consisting of administering the composition in three separate doses; and (ii)(b) if the age is at least 6 months and less than about 9 years, administering to the subject a composition comprising Norovirus virus-like particles (VLPs) in a dosing regimen consisting of administering the composition in two separate doses. In further embodiments, the composition is administered in a dosing regimen consisting of administering the composition in two separate doses if the age is at least 6 months and less than bout 8 years, less than about 7 years, less than about 6 years, less than about 5 years, less than about 4 years, less than about 3 years, less than about 2 years, or less than about 1 year.

### BRIEF DESCRIPTION OF THE DRAWINGS

FIG. 1 is a schematic of the design of a phase 2 clinical study in pediatric patients.

FIG. 2A-2D show the seroresponse rate (SRR) to GI.1 and GII.4 Norovirus, measured by pan-Ig titer. In each bar graph, from left to right, the four dosing groups are: 15/15; 15/50; 50/50; and 50/150. The 1-dose groups of Cohort 1 (Vaccine/Placebo; V/P) are shown in the top row of bar graphs (FIG. 2A). The two-dose groups of Cohort 1 (Vaccine/Vaccine; V/V) are shown in the second row of bar graphs (FIG. 2B). The 2-dose group of Cohort 2 (Vaccine/Vaccine/Placebo; V/V/P) is shown in the third row (FIG. 2C). The 3-dose group of Cohort 2 (Vaccine/Vaccine/Vaccine; V/V/V) is shown in the fourth row (FIG. 2D). A seroresponse was a ≥4-fold rise in the titer (at Day 57 (28 days post dose 2) or Day 140 (28 days post dose 3). In the figure, Group 1 (4 to <9 years) is shown as 4-8 y; Groups 2 and 2a (12 months to <4 years) are shown as 1-3 y; Group 3 (6 months to <12 months) is shown as 6-11 mo; and Group 4 (6 weeks to 6 months) is shown as 6-25 wk.

FIG. 3A-3D show the SRR to GI.1 and GII.4 Norovirus, measured by Histo-blood group antigen (HBGA)-blocking titers. In each bar graph, from left to right, the four dosing groups are: 15/15; 15/50; 50/50; and 50/150. The 1-dose groups of Cohort 1 (V/P) are shown in the top row of bar graphs (FIG. 3A). The two-dose groups of Cohort 1 (V/V) are shown in the second row of bar graphs (FIG. 3B). The 2-dose group of Cohort 2 (V/V/P) is shown in the third row (FIG. 3C). The 3-dose group of Cohort 2 (V/V/V) is shown in the fourth row (FIG. 3D). A seroresponse was a ≥4-fold rise in the titer (at Day 57 (28 days post dose 2) or Day 140 (28 days post dose 3). In the figure, Group 1 (4 to <9 years) is shown as 4-8 y; Groups 2 and 2a (12 months to <4 years) are shown as 1-3 y; Group 3 (6 months to <12 months) is shown as 6-11 mo; and Group 4 (6 weeks to 6 months) is shown as 6-25 wk.

FIG. 4A-4D show the GI.1-specific HBGA-blocking Geometric Mean Titers (GMTs) by age group and arm. In each graph, from left to right, the four dosing groups are: 15/15; 15/50; 50/50; and 50/150. The 1-dose groups of Cohort 1 (V/P) are shown in the top row of bar graphs (FIG. 4A). The two-dose groups of Cohort 1 (V/V) are shown in the second row of bar graphs (FIG. 4B). The 2-dose group of Cohort 2 (V/V/P) is shown in the third row (FIG. 4C). The 3-dose group of Cohort 2 (V/V/V) is shown in the fourth row (FIG. 4D). The GMT was adjusted with respect to the baseline titers (Day 1) using an analysis of covariance (ANCOVA) model.

FIG. 5A-5D show the GII.4c-specific HBGA-blocking GMTs by age group and arm. In each graph, from left to right, the four dosing groups are: 15/15; 15/50; 50/50; and 50/150. The 1-dose groups of Cohort 1 (V/P) are shown in the top row of bar graphs (FIG. 5A). The two-dose groups of Cohort 1 (V/V) are shown in the second row of bar graphs (FIG. 5B). The 2-dose group of Cohort 2 (V/V/P) is shown in the third row (FIG. 5C). The 3-dose group of Cohort 2 (V/V/V) is shown in the fourth row (FIG. 5D). The GMT was adjusted with respect to the baseline titers (Day 1) using an analysis of covariance (ANCOVA) model.

FIG. 6A-6B show the GMTs for GI.1-specific IgA (FIG. 6A) and GII.4c-specific IgA (FIG. 6B) for Group 1 (4 to <9 years). Patients in the 2-dose group are shown with a solid line and patients in the 1-dose group are shown with a dotted line (for measurements taken after placebo administration at the Dose 2 timepoint).

FIG. 7A-7B show the GMTs for GI.1-specific IgA (FIG. 7A) and GII.4c-specific IgA (FIG. 7B) for Group 2 (1 to <4 years). Patients in the 2-dose group are shown with a solid line and patients in the 1-dose group are shown with a dotted line (for measurements taken after placebo administration at the Dose 2 timepoint).

FIG. 8A-8B show the GMTs for GI.1-specific IgA (FIG. 8A) and GII.4c-specific IgA (FIG. 8B) for Group 3 (6 to <12 months). Patients in the 2-dose group are shown with a solid line and patients in the 1-dose group are shown with a dotted line (for measurements taken after placebo administration at the Dose 2 timepoint).

FIG. 9A-9B show the GMTs for GI.1-specific IgA (FIG. 9A) and GII.4c-specific IgA (FIG. 9B) for Group 4 (6 weeks to <6 months). Patients in the 3-dose group are shown with a solid line and patients in the 2-dose group are shown with a dotted line (for measurements taken after placebo administration at the Dose 3 timepoint).

FIG. 10 shows the cross-reactivity to other Norovirus strains after administration of two doses of bivalent GI.1/GII.4c Norovirus vaccine to Group 2 subjects (1 to <4 years).

FIG. 11A-11B show the cross-reactivity to other Norovirus strains after administration of bivalent GI.1/GII.4c Norovirus vaccine to Group 3 subjects (6 to <12 months). FIG. 11A shows the cross-reactivity after the first dose. FIG. 11B shows higher cross-reactivity after the second dose.

FIG. 12A-12B provide an overview of the solicited local AEs after each dose in the clinical study, by group. The % of subjects who experienced the indicated AEs at the indicated intensities (mild, moderate, severe) are shown for Group 1 (top row, FIG. 12A), Group 2 (second row from the top, FIG. 12A), Group 2a (third row from the top, FIG. 12A), and Group 3 (bottom row, FIG. 12A). FIG. 12B shows the % of subjects who experienced the indicated AEs at the indicated intensities for Group 4. Solicited local AEs were recorded within 7 days after each dose. The percentages were calculated as 100×number of subjects/N_{T} for all local AEs and 100×number of subjects/N_{S} for individual AE symptom, where N_{T}=number of subjects in the Safety set and N_{S}=number of subjects in the Safety set evaluated for that symptom. The percentages annotated in the graphs have been rounded in accordance with the “round half to the nearest even integer” convention.

FIG. 13A-13C provide an overview of the solicited systemic AEs after each dose in the clinical study, by group. The % of subjects who experienced the indicated AEs at the indicated intensities are shown for Group 1 (top row, FIG. 13A), Group 2 (bottom row, FIG. 13A), Group 2a (top row, FIG. 13B), and Group 3 (bottom row, FIG. 13B). FIG. 13C shows the % of subjects who experienced the indicated AEs at the indicated intensities for Group 4. Solicited systemic AEs were recorded within 7 days after each dose. Fever was considered as a solicited systemic AE; and for the graphs, fever was graded as mild, 38.0 to <38.5° C.; moderate, 38.5 to <39.0° C.; and severe, ≥39° C. (however, the fever intensity grading is depicted as mild in the intensity grading for AEs overall). The percentages were calculated as 100×number of subjects/N_{T} for all systemic AEs and 100×number of subjects/N_{S} for individual AE symptom, where N_{T}=number of subjects in the Safety set and N_{S}=number of subjects in the Safety set evaluated for that symptom. The percentages annotated in the graphs have been rounded in accordance with the “round half to the nearest even integer” convention.

### DETAILED DESCRIPTION OF THE INVENTION

The present invention relates to methods of eliciting protective immunity to Norovirus infections in a subject, wherein the subject is a pediatric human subject. In particular, the present invention provides methods of eliciting protective immunity against Norovirus in a pediatric subject by parenterally administering to the subject at least two doses of a vaccine comprising Norovirus VLPs. The inventors have surprisingly discovered that a composition comprising Norovirus VLPs can be effectively and safely administered to pediatric subjects and elicit protective immunity against Norovirus infection in those subjects. Administration of two or three doses of a vaccine composition comprising Norovirus VLPs to human pediatric subjects induced a rapid, robust serum conversion (e.g., at least a three-fold increase in antigen-specific serum antibody titers above pre-vaccination levels) that is indicative of a protective immune response against Norovirus infection and illness. The pediatric subjects are as young as 6 weeks of age and the elicitation of the protective immune response was achieved in connection with an acceptable safety profile.

Dosing regimens comprising 15 μg to 150 μg of each VLP type administered according to the methods provided herein were safe and effective in pediatric subjects. In an embodiment, the vaccine composition administered to the pediatric subject comprises about 50 μg of a Norovirus genogroup I, genotype 1 (GI.1) VLP and about 150 μg of a Norovirus genogroup II, genotype 4 (GII.4) VLP that is generated from a consensus sequence of different GII.4 strains. In a further embodiment, the GII.4 VLP comprises a capsid protein according to SEQ ID NO: 1. In a yet further embodiment, the composition further comprises 500 μg aluminum hydroxide.

The invention provides a vaccine composition comprising one or more Norovirus antigens. By “Norovirus,” “Norovirus (NOR),” “norovirus,” and grammatical equivalents herein, are meant members of the genus Norovirus of the family Caliciviridae. In some embodiments, a Norovirus can include a group of related, positive-sense single-stranded RNA, nonenveloped viruses that can be infectious to human or non-human mammalian species. In some embodiments, a Norovirus can cause acute gastroenteritis in humans. Noroviruses also can be referred to as small round structured viruses (SRSVs) having a defined surface structure or ragged edge when viewed by electron microscopy.

Included within the Noroviruses are at least five genogroups (GI, GII, GIII, GIV, and GV). GI, GII, and GIV Noroviruses are infectious in humans, while GIII Noroviruses primarily infect bovine species. GV has recently been isolated from mice (Zheng et al. (2006) Virology, Vol 346: 312-323). Representative of GIII are the Jena and Newbury strains, while the Alphatron, Fort Lauderdale, and Saint Cloud strains are representative of GIV. The GI and Gil groups may be further segregated into genetic clusters or genotypes based on genetic classification (Ando et al. (2000) J. Infectious Diseases, Vol. 181(Supp2):5336-5348; Lindell et al. (2005) J. Clin. Microbiol., Vol. 43(3): 1086-1092) and/or classification based on outbreaks or epidemics. As used herein, the term genetic clusters is used interchangeably with the term genotypes. Within genogroup I, there are 8 GI clusters known to date (with prototype virus strain name): GI.1 (Norwalk (NV-USA93)); GI.2 (Southhampton (SOV-GBR93)); GI.3 (Desert Shield (DSV-USA93), or GI.3.2000); GI.4 (Cruise Ship virus/Chiba (Chiba-JPN00), or GI.4.2000); GI.5 (318/Musgrove (Musgrov-GBROO)); GI.6 (Hesse (Hesse-DEU98)); GI.7 (Wnchest-GBROO); and GI.8 (Boxer-USA02). Within genogroup II, there are 19 GII clusters known to date (with prototype virus strain name): GII.1 (Hawaii (Hawaii-USA94)); GII.2 (Snow Mountain/Melksham (Msham-GBR95)); GII.3 (Toronto (Toronto-CAN93), or GII.3.1999); GII.4 (Bristol/Lordsdale (Bristol-GBR93), GII.4.2006b, or GII.4.2012); GII.5 (290/Hillingdon (Hilingd-GBROO)); GII.6 (269/Seacroft (Seacrof-GBROO)); GII.7 (273/Leeds (Leeds-GBROO)); GII.8 (539/Amsterdam (Amstdam-NLD99)); GII.9 (378 (VABeach-USA01)), GII.10 (Erfurt-DEU01); GII.11 (SW9180JPN01); GII.12 (Wortley-GBROO); GII.13 (Faytvil-USA02); GII.14 (M7-USA03); GII.15 (J23-USA02); GII.16 (Tiffin-USA03); GII.17 (CSE1-USA03), or GII.17.2015; GII.18 (QW101/2003/US) and GII.19 (QW170/2003/US).

By “Norovirus” also herein is meant recombinant Norovirus virus-like particles (rNOR VLPs). In some embodiments, recombinant expression of at least the Norovirus capsid protein encoded by ORF2 in cells, e.g., from a baculovirus vector in 519 cells, can result in spontaneous self-assembly of the capsid protein into VLPs. In some embodiments, recombinant expression of at least the Norovirus proteins encoded by ORF1 and ORF2 in cells, e.g., from a baculovirus vector in Sf9 cells, can result in spontaneous self-assembly of the capsid protein into VLPs. VLPs are structurally similar to Noroviruses but lack the viral RNA genome and therefore are not infectious. Accordingly, “Norovirus” includes virions that can be infectious or non-infectious particles, which include defective particles.

Examples of Noroviruses are generally known in the art and include those disclosed, for example, in U.S. Publication Nos. US2013-0273102 and US2011-0195113, the entire contents of each of which are hereby incorporated by reference As new strains are identified and their genetic sequences are made available, one skilled in the art would be able to employ VLPs using these contemporary strains in the compositions and methods of the present invention using ordinary skill. Thus, the present disclosure encompasses administering VLPs made from such strains as suitable antigens for use in the compositions and methods for eliciting protective immunity in pediatric subjects as described herein.

Norovirus antigens in vaccine compositions may be in the form of peptides, proteins, or virus-like particles (VLPs). In a preferred embodiment, the Norovirus antigen comprises VLPs. As used herein, “virus-like particle(s)” or “VLPs” refer to a virus-like particle(s), fragment(s), aggregate(s), or portion(s) thereof produced from the capsid protein coding sequence of Norovirus and comprising antigenic characteristic(s) similar to those of infectious Norovirus particles. Norovirus antigens may also be in the form of capsid monomers, capsid multimers, protein or peptide fragments of VLPs, or aggregates or mixtures thereof. The Norovirus antigenic proteins or peptides may also be in a denatured form, produced using methods known in the art.

The VLPs of the present invention can be formed from either the full length Norovirus capsid protein such as VP1 and/or VP2 proteins or certain VP1 or VP2 derivatives using standard methods in the art. Alternatively, the capsid protein used to form the VLP is a truncated capsid protein. In some embodiments, for example, at least one of the VLPs comprises a truncated VP1 protein. In other embodiments, all the VLPs comprise truncated VP1 proteins. The truncation may be an N- or C-terminal truncation. Truncated capsid proteins are suitably functional capsid protein derivatives. Functional capsid protein derivatives are capable of raising an immune response (if necessary, when suitably adjuvanted) in the same way as the immune response is raised by a VLP consisting of the full length capsid protein. In some embodiments, the compositions provided herein for use in the disclosed methods include truncated capsid proteins. In other embodiments, the compositions provided herein for use in the disclosed methods have been purified such that they do not include truncated capsid proteins, or include 40%, 30%, 20%, 10%, 5%, 1%, or less truncated capsid proteins.

VLPs may contain major VP1 proteins and/or minor VP2 proteins. In some embodiments, each VLP contains VP1 and/or VP2 protein from only one Norovirus genogroup giving rise to a monovalent VLP. As used herein, the term “monovalent” means the antigenic proteins are derived from a single Norovirus genogroup. For example, the VLPs contain VP1 and/or VP2 from a virus strain of genogroup I (e.g., VP1 and VP2 from Norwalk virus) or a consensus sequence of genogroup I strains; or the VLPs contain VP1 and/or VP2 from a virus strain of genogroup II (e.g, VP1 and/or VP2 from a GII.4 strain) or a consensus sequence of GII.4 strains (e.g., GII.4c, SEQ ID NO: 1 herein). Preferably the VLP is comprised of predominantly VP1 proteins.

In one embodiment of the invention, the composition comprises a mixture of monovalent VLPs wherein the composition includes VLPs comprised of VP1 and VP2 from a single Norovirus genogroup mixed with VLPs comprised of VP1 and VP2 from a different Norovirus genogroup (e.g. Norwalk virus and Houston virus) taken from multiple viral strains. Purely by way of example the composition can contain monovalent VLPs from one or more strains of Norovirus genogroup I together with monovalent VLPs from one or more strains of Norovirus genogroup II. Strains may be selected based on their predominance of circulation at a given time. In certain embodiments, the Norovirus VLP mixture is composed of GI.1 and GII.4 viral strains. More preferably, the Norovirus VLP mixture is composed of the strains of Norwalk and a consensus capsid sequence derived from genogroup II Noroviruses. Consensus capsid sequences derived from circulating Norovirus sequences and VLPs made with such sequences are described in WO 2010/017542, which is herein incorporated by reference in its entirety. For instance, in one embodiment, a consensus capsid sequence derived from genogroup II, genotype 4 (GII.4) viral strains comprises a sequence of SEQ ID NO: 1. This VLP is referred to herein as “GII.4c.” Thus, in some embodiments, the vaccine composition comprises a mixture of monovalent VLPs, wherein one monovalent VLP comprises a capsid protein from a genogroup I Norovirus (e.g. Norwalk) and the other monovalent VLP comprises a consensus capsid protein comprising a sequence of SEQ ID NO: 1 (GII.4c).

The combination of VLPs within the composition preferably does not reduce the immunogenicity of each VLP type. In particular it is preferred that there is no interference between Norovirus VLPs in the combination of the invention, such that the combined VLP composition of the invention is able to elicit immunity against infection by each Norovirus genotype and/or genogroup represented in the vaccine. In embodiments, the immune response against a given VLP type in the combination is at least 50% of the immune response of that same VLP type when measured individually, preferably 100% or substantially 100%. Moreover, the claimed compositions may induce cross-reactivity to virus strains not present in the composition. For example, the claimed compositions may induce cross-reactivity to other Norovirus genotypes and/or other Norovirus genogroups that are not represented in the composition. A composition or method that induces cross-reactivity may induce an immune response to the other virus strain; and/or may elicit protective immunity in a subject against the other virus strain.

For example, administration of the compositions provided herein to subjects may induce immunity against other genogroup I strains (other than GI.1); other genogroup II strains (other than the strains making ups GII.4c); and/or other genogroups (other than genogroup I and genogroup II). By way of nonlimiting example, the compositions provided herein may induce immunity against one or more strains selected from GII.4.2006b, GII.4.2012, GI.3.2000, GI.4.2000, GII.3.1999, and GII.17.2015. In some embodiments, the compositions provided herein may induce immunity against GII.4.2006b. In some embodiments, the compositions provided herein may induce immunity against GII.4.2012. The immune response may suitably be measured, for example, by antibody responses, as illustrated in the examples herein.

Multivalent VLPs may be produced by separate expression of the individual capsid proteins followed by combination to form VLPs. Alternatively, multiple capsid proteins may be expressed within the same cell, from one or more DNA constructs. For example, multiple DNA constructs may be transformed or transfected into host cells, each vector encoding a different capsid protein. Alternatively a single vector having multiple capsid genes, controlled by a shared promoter or multiple individual promoters, may be used. IRES elements may also be incorporated into the vector, where appropriate. Using such expression strategies, the co-expressed capsid proteins may be co-purified for subsequent VLP formation, or may spontaneously form multivalent VLPs which can then be purified. A preferred process for multivalent VLP production comprises preparation of VLP capsid proteins or derivatives, such as VP1 proteins, from different Norovirus genotypes, mixing the proteins, and assembly of the proteins to produce multivalent VLPs. The VP1 proteins may be in the form of a crude extract, be partially purified or purified prior to mixing. Assembled monovalent VLPs of different genogroups may be disassembled, mixed together and reassembled into multivalent VLPs. Preferably the proteins or VLPs are at least partially purified before being combined. Optionally, further purification of the multivalent VLPs may be carried out after assembly.

Where multivalent VLPs are used, preferably the components of the VLPs are mixed in the proportions in which they are desired in the final mixed VLP. For example, a mixture of the same amount of a partially purified VP1 protein from Norwalk and Houston viruses (or other Norovirus strains) provides a multivalent VLP with approximately equal amounts of each protein. Compositions comprising multivalent VLPs may be stabilized by solutions known in the art, such as those of WO 98/44944, WO 00/45841, incorporated herein by reference.

Compositions of the invention may comprise other proteins or protein fragments in addition to Norovirus VP1 and VP2 proteins or derivatives. Other proteins or peptides may also be co-administered with the composition of the invention. Optionally, the composition may also be formulated or co-administered with non-Norovirus antigens. Suitably these antigens can provide protection against other diseases.

The VP1 protein or functional protein derivative is suitably able to form a VLP, and VLP formation can be assessed by standard techniques such as, for example, size exclusion chromatography, electron microscopy and dynamic laser light scattering.

The antigenic molecules of the present invention can be prepared by isolation and purification from the organisms in which they occur naturally, or they may be prepared by recombinant techniques. Preferably, the Norovirus VLP antigens are prepared from insect cells such as Sf9 or H5 cells, although any suitable cells such as E. coli or yeast cells, for example, S. cerevisiae, S. pombe, Pichia pastori or other Pichia expression systems, or mammalian cell expression such as CHO or HEK systems may also be used. When prepared by a recombinant method or by synthesis, one or more insertions, deletions, inversions or substitutions of the amino acids constituting the peptide may be made. Each of the aforementioned antigens is preferably used in the substantially pure state.

The procedures of production of norovirus VLPs in insect cell culture have been previously disclosed in U.S. Pat. No. 6,942,865, which is incorporated herein by reference in its entirety. Briefly, a cDNA from the 3′ end of the genome containing the viral capsid gene (ORF2) and a minor structural gene (ORF3) is cloned. The recombinant baculoviruses carrying the viral capsid genes is constructed from the cloned cDNAs. Norovirus VLPs are produced in Sf9 or H5 insect cell cultures.

In some embodiments, the vaccine composition comprises one or more adjuvants in combination with the Norovirus antigen. Adjuvants such as aluminum hydroxide or mineral oil contain a substance designed to protect the antigen from rapid catabolism. Suitable adjuvants are commercially available as, for example, Freund's Incomplete Adjuvant and Complete Adjuvant (Pifco Laboratories, Detroit, Mich.); Merck Adjuvant 65 (Merck and Company, Inc., Rahway, N.J.); aluminum salts such as aluminum hydroxide ((Al(OH)₃), aluminum hydroxide gel (alum) or aluminum phosphate; salts of calcium, iron or zinc; an insoluble suspension of acylated tyrosine acylated sugars; cationically or anionically derivatized polysaccharides; polyphosphazenes; biodegradable microspheres; and Quil A. In some embodiments, the adjuvant is aluminum hydroxide (Al(OH)₃). In further embodiments, the adjuvant is aluminum hydroxide, and is present in each composition in an amount of about 10 μg, about 25 μg, about 50 μg, about 75 μg, about 100 μg, about 125 μg, about 150 μg, about 175 μg, about 200 μg, about 225 μg, about 50 μg, about 275 μg, about 300 μg, about 350 μg, about 375 μg, about 400 μg, about 425 μg, about 450 μg, about 475 μg, about 500 μg, about 525 μg, about 550 μg, about 575 μg, about 600 μg, about 625 μg, about 650 μg, about 675 μg, about 700 μg, about 750 μg, about 800 μg, about 850 μg, about 900 μg, about 950 μg, or about 1000 μg. In certain embodiments, the composition comprises aluminum hydroxide in an amount of about 500 μg.

Suitable adjuvants also include, but are not limited to, toll-like receptor (TLR) agonists, particularly toll-like receptor type 4 (TLR-4) agonists (e.g., monophosphoryl lipid A (MPL), synthetic lipid A, lipid A mimetics or analogs), aluminum salts, cytokines, saponins, muramyl dipeptide (MDP) derivatives, CpG oligos, lipopolysaccharide (LPS) of gram-negative bacteria, polyphosphazenes, emulsions, virosomes, cochleates, poly(lactide-co-glycolides) (PLG) microparticles, poloxamer particles, microparticles, liposomes, oil-in-water emulsions, MF59, and squalene. In some embodiments, the adjuvants are not bacterially-derived exotoxins. Preferred adjuvants include adjuvants which stimulate a Thl type response such as 3DMPL or QS21.

In some embodiments, the vaccine composition comprises two adjuvants. A combination of adjuvants may be selected from those described above. In one particular embodiment, the two adjuvants are MPL and aluminum hydroxide (e.g., alum). In another particular embodiment, the two adjuvants are MPL and oil. In preferred embodiments, the vaccine composition comprises a single adjuvant. In further embodiments, the single adjuvant is aluminum hydroxide.

The term “effective adjuvant amount” or “effective amount of adjuvant” will be well understood by those skilled in the art, and includes an amount of one or more adjuvants which is capable of stimulating the immune response to an administered antigen, i.e., an amount that increases the immune response of an administered antigen composition, as measured in terms of the IgA levels in the nasal washings, serum IgG or IgM levels, or B and T-Cell proliferation. Suitably effective increases in immunoglobulin levels include by more than 5%, preferably by more than 25%, and in particular by more than 50%, as compared to the same antigen composition without any adjuvant.

In one embodiment, the present invention provides a vaccine composition formulated for parenteral administration, wherein the composition includes at least two types of Norovirus VLPs in combination with aluminum hydroxide and a buffer. The buffer can be selected from the group consisting of L-histidine, imidazole, succinic acid, tris, citric acid, bis-tris, pipes, mes, hepes, glycine amide, and tricine. In one embodiment, the buffer is L-histidine or imidazole. Preferably, the buffer is present in a concentration from about 15 mM to about 50 mM, more preferably from about 18 mM to about 40 mM, or most preferably about 20 mM to about 25 mM. In some embodiments, the pH of the antigenic or vaccine composition is from about 6.0 to about 7.0, or from about 6.2 to about 6.8, or about 6.5. The vaccine composition can be an aqueous formulation. In some embodiments, the vaccine composition is a lyophilized powder and reconstituted to an aqueous formulation.

In certain embodiments, the vaccine composition can comprise about 15 μg to about 150 μg of each Norovirus VLP, more preferably about 15 μg to about 50 μg of a genogroup I VLP and about 50 μg to about 150 μg of a genogroup II VLP. Thus, in some embodiments, the dose of one type of Norovirus VLP is different than the dose of the other type of Norovirus VLP. For instance, in certain embodiments, the vaccine composition comprises about 15 μg of a genogroup I VLP and about 50 μg of a genogroup II VLP. In certain embodiments, the vaccine composition comprises about 50 μg of a genogroup I VLP and about 150 μg of a genogroup II VLP. In other embodiments, the vaccine composition comprises about 15 μg of a genogroup I VLP and about 15 μg of a genogroup II VLP; or about 50 μg of a genogroup I VLP and about 50 μg of a genogroup II VLP. In certain embodiments, a vaccine composition of the invention for eliciting a protective immune response against Norovirus in human pediatric subjects comprises 50 μg GI.1 VLP and 150 μg GII.4c VLP and 500 μg aluminum hydroxide.

In some embodiments, the vaccine compositions further comprise a pharmaceutically acceptable salt, including, but not limited to, sodium chloride, potassium chloride, sodium sulfate, amonium sulfate, and sodium citrate. In one embodiment, the pharmaceutically acceptable salt is sodium chloride. The concentration of the pharmaceutically acceptable salt can be from about 10 mM to about 200 mM, with preferred concentrations in the range of from about 100 mM to about 150 mM. Preferably, the vaccine compositions of the invention contain less than 2 mM of free phosphate. In some embodiments, the vaccine compositions comprise less than 1 mM of free phosphate. The vaccine compositions may also further comprise other pharmaceutically acceptable excipients, such as sugars (e.g., sucrose, trehalose, mannitol) and surfactants.

As discussed herein, the compositions of the invention can be formulated for administration as vaccines formulations. As used herein, the term “vaccine” refers to a formulation which contains Norovirus VLPs or other Norovirus antigens of the present invention as described above, which is in a form that is capable of being administered to a human, preferably a pediatric human, and which induces a protective immune response sufficient to induce immunity to prevent and/or ameliorate a Norovirus infection or Norovirus-induced illness and/or to reduce at least one symptom of a Norovirus infection or illness.

As used herein, the term “immune response” refers to both the humoral immune response and the cell-mediated immune response. The humoral immune response involves the stimulation of the production of antibodies by B lymphocytes that, for example, neutralize infectious agents, block infectious agents from entering cells, block replication of said infectious agents, and/or protect host cells from infection and destruction. The cell-mediated immune response refers to an immune response that is mediated by T-lymphocytes and/or other cells, such as macrophages, against an infectious agent, exhibited by a vertebrate (e.g., a human), that prevents or ameliorates infection or reduces at least one symptom thereof. In particular, “protective immunity” or “protective immune response” refers to immunity or eliciting an immune response against an infectious agent, which is exhibited by a vertebrate (e.g., a human), that prevents or ameliorates an infection or reduces at least one symptom thereof. Specifically, induction of a protective immune response from administration of the vaccine is evident by elimination or reduction of the presence of one or more symptoms of acute gastroenteritis or a reduction in the duration or severity of such symptoms. Clinical symptoms of gastroenteritis from Norovirus include nausea, diarrhea, loose stool, vomiting, fever, and general malaise. A protective immune response that reduces or eliminates disease symptoms will reduce or stop the spread of a Norovirus outbreak in a population. Vaccine preparation is generally described in Vaccine Design (“The subunit and adjuvant approach” (eds Powell M. F. & Newman M. J.) (1995) Plenum Press New York). The compositions of the present invention can be formulated, for example, for delivery to one or more of the oral, gastro-intestinal, and respiratory (e.g. nasal) mucosa. The compositions of the present invention can be formulated, for example, for delivery by injection, such as parenteral injection (e.g., intravenous, subcutaneous, intradermal, or intramuscular injection).

Where the composition is intended for parenteral injection, such as intravenous (i.v.), subcutaneous (s.c.), intradermal, or intramuscular (i.m.) injection, it is typically formulated as a liquid suspension (i.e. aqueous formulation) comprised of at least one type of Norovirus VLP and optionally at least one adjuvant. In a preferred embodiment, a parenterally-formulated (e.g., i.m., i.v., or s.c.-formulated) liquid vaccine comprises Norovirus genogroup I and/or genogroup II VLPs and aluminum hydroxide. In a further preferred embodiment, the composition is formulated for i.m. injection.

The vaccine compositions hereinbefore described may be lyophilized and stored in an anhydrous form until they are ready to be used, at which point they are reconstituted with diluent. Alternatively, different components of the composition may be stored separately in a kit (any or all components being lyophilized). The components may remain in lyophilized form for dry formulation or be reconstituted for liquid formulations, and either mixed prior to use or administered separately to the patient. In some embodiments, the vaccine compositions are stored in kits in liquid formulations and may be accompanied by delivery devices, such as syringes equipped with needles. In other embodiments, the liquid vaccine compositions may be stored within the delivery devices in a kit. For example, a kit may comprise pre-filled syringes, autoinjectors, or injection pen devices containing a liquid formulation of a vaccine composition described herein.

The composition is administered to a pediatric patient in an amount sufficient to elicit an immune response to the specific antigens and/or to prevent, alleviate, reduce, or cure symptoms and/or complications from the disease or infection, and thus reduce or stop the spread of a Norovirus outbreak in a population. An amount adequate to accomplish this is defined as a “therapeutically effective dose.” The amount of antigen in each vaccine composition is selected as an amount which induces a robust immune response without significant, adverse side effects. The inventors of the present disclosure unexpectedly found that a multiple-dose regimen of 50 μg GI.1 and 150 μg GII.4c VLPs safely elicited protective immunity against norovirus infection in pediatric subjects, including subjects as young as 6 weeks of age. For example, three doses of the 50 μg/150 μg (GI.1/GII.4c) formulation elicited protective immunity in subjects aged 6 weeks to <6 months; and two doses of the 50 μg/150 μg (GI.1/GII.4c) formulation elicited protective immunity in subjects aged 6 months to <9 years.

The methods and uses comprising administration of the compositions provided herein may be associated with a surprisingly save adverse event profile in a pediatric population. As demonstrated in the Example and Figures disclosed herewith, the incidence of adverse events was within acceptable limits for each of the cohorts and age groups tested. In particular, the serious adverse event (SAE) occurrence was negligible for many of the groups and there were no vaccine-related SAEs or fatal SAEs reported in the study described in Example 1. Therefore, in some embodiments, the present methods may be performed in the relative absence of vaccine-related serious adverse events. In some embodiments, the safety profile of the present compositions is safer and/or associated with a statistically significant lower adverse event incidence than a comparable vaccine composition. Comparable vaccine compositions may be those targeting a virus that causes gastroenteritis. For example, a comparable vaccine composition may be one targeting rotavirus. Another comparable vaccine composition may be one targeting adenovirus. Another comparable vaccine composition may be one targeting influenza virus.

The present invention provides methods for eliciting protective immunity against Norovirus in pediatric subjects. As used herein, “pediatric subjects” and “pediatric patients” and the like refer to infants aged 6 weeks or older; and children aged 9 years or younger. In some embodiments, “infants” may refer to human subjects less than 6 months of age; or aged between 6 weeks and <6 months.

In some embodiments, the vaccine composition induces at least a three-fold increase in Norovirus-specific serum antibody titer as compared to the titer in the subject prior to administration of the composition. In some embodiments, the vaccine composition induces at least a four-fold increase in Norovirus-specific serum antibody titer as compared to the titer in the subject prior to administration of the composition. In some embodiments, the vaccine composition induces at least a five-fold increase in Norovirus-specific serum antibody titer as compared to the titer in the subject prior to administration of the composition. In some embodiments, the vaccine composition induces at least a six-fold increase in Norovirus-specific serum antibody titer as compared to the titer in the subject prior to administration of the composition. In other embodiments, the vaccine composition induces a Norovirus-specific serum antibody titer comparable to the antibody titer induced by exposure to live Norovirus in a natural infection—i.e., a greater than ten-fold increase in Norovirus-specific serum antibody as compared to the titer in the human subject prior to administration of the composition. In certain embodiments, the vaccine composition induces the increase in Norovirus-specific serum antibody titer within seven days of administration of the composition. Preferably, the vaccine composition is administered by an intravenous, subcutaneous, or intramuscular route of administration. In a certain embodiment, the vaccine composition is administered intramuscularly to the human subject.

In one embodiment of the method, the subject is a human pediatric subject and the vaccine confers protection from one or more symptoms of Norovirus infection. The pediatric subject may be about 6 weeks of age to about 9 years of age.

Because Norovirus is not able to be cultured in vitro, no viral neutralization assays are currently available. A functional assay which serves as a substitute for the neutralization assay is the hemagglutination inhibition (HAI) assay. HAI measures the ability of Norovirus vaccine-induced antibodies to inhibit the agglutination of antigen-coated red blood cells by Norovirus VLPs because Norovirus VLPs bind to red blood cell antigens (e.g. histo-blood group antigens). This assay is also known as a carbohydrate blocking assay, as it is indicative of the functional ability of antibodies to block binding of the virus or VLPs to blood group antigen carbohydrates on a red blood cell. In this assay, a fixed amount of Norovirus VLPs is mixed with a fixed amount of red blood cells and serum from immunized subjects. If the serum sample contains functional antibodies, the antibodies will bind to the VLPs, thereby inhibiting the agglutination of the red blood cells. As used herein, “functional antibodies” refer to antibodies that are capable of inhibiting the interaction between Norovirus particles and red blood cell antigens. In other words, functional antibody titer is equivalent to histo-blood group antigen (HBGA) or carbohydrate blocking antibody titer. The serum titer of Norovirus-specific functional antibodies can be measured by the HAI assay described above. The serum titer of Norovirus-specific functional antibodies can also be measured using an ELISA-based assay in which a carbohydrate H antigen is bound to microtiter wells and Norovirus VLP binding to H antigen is detected in the presence of serum (see Reeck et al. (2010) J Infect Dis, Vol. 202(8):1212-1218). An increase in the level of Norovirus-specific functional antibodies can be an indicator of a protective immune response. Thus, in one embodiment, the administration of the vaccine elicits a protective immunity comprising an increase in the serum titer of Norovirus-specific functional antibodies as compared to the serum titer in a human not receiving the vaccine. The serum titer of Norovirus-specific functional antibodies indicative of a protective immune response is preferably a geometric mean titer greater than 40, 50, 75, 100, 125, 150, 175, 200 as measured by the HAI assay or blocking titer (BT)₅₀ (50% inhibition of H antigen binding by Norovirus VLPs) geometric mean titer of greater than 100, 150, 200, 250, 300, 350, 400, 450, or 500 as measured by the H antigen binding assay. In one embodiment, the serum titer of Norovirus-specific functional antibodies is a geometric mean titer greater than 40 as measured by the HAI assay. In another embodiment, the serum titer of Norovirus-specific functional antibodies is a geometric mean titer greater than 100 as measured by the HAI assay. In another embodiment, the serum titer of Norovirus-specific functional antibodies is a BT₅₀ geometric mean titer greater than 100 as measured by the H antigen binding assay. In still another embodiment, the serum titer of Norovirus-specific functional antibodies is a BT₅₀ geometric mean titer greater than 200 as measured by the H antigen binding assay.

In a further aspect, the administration of the vaccine elicits a protective immune response comprising an IgA mucosal immune response and an IgG systemic immune response by administering parenterally (preferably intramuscularly) to the pediatric subject at least two doses of an antigenic or vaccine composition comprising one or more types of Norovirus antigens and optionally at least one effective adjuvant (e.g., aluminum hydroxide). The inventors have surprisingly found that parenteral administration of the Norovirus vaccine compositions described herein induces a robust IgA response in children, in addition to a strong IgG response. Typically, strong IgA responses are only observed when vaccines are administered through a mucosal route of administration.

As mentioned above, administration of a vaccine composition of the present invention prevents and/or reduces at least one symptom of Norovirus infection. Symptoms of Norovirus infection are well known in the art and include nausea, vomiting, diarrhea, and stomach cramping. Additionally, a patient with a Norovirus infection may have a low-grade fever, headache, chills, muscle aches, and fatigue. The invention also encompasses a method of inducing a protective immune response in a subject experiencing a Norovirus infection by administering to the subject a vaccine formulation of the invention such that at least one symptom associated with the Norovirus infection is alleviated and/or reduced. A reduction in a symptom may be determined subjectively or objectively, e.g., self assessment by a subject, by a clinician's assessment or by conducting an appropriate assay or measurement (e.g. body temperature), including, e.g., a quality of life assessment, a slowed progression of a Norovirus infection or additional symptoms, a reduced severity of Norovirus symptoms or suitable assays (e.g. antibody titer, RT-PCR antigen detection, and/or B-cell or T-cell activation assay). An effective response may also be determined by directly measuring (e.g., RT-PCR) virus load in stool samples, which reflects the amount of virus shed from the intestines). The objective assessment comprises both animal and human assessments.

The invention also provides a method of generating antibodies to one or more Norovirus antigens, said method comprising administration of a vaccine composition of the invention as described above to a subject. These antibodies can be isolated and purified by routine methods in the art. The isolated antibodies specific for Norovirus antigens can be used in the development of diagnostic immunological assays. These assays could be employed to detect a Norovirus in clinical samples and identify the particular virus causing the infection (e.g. Norwalk, Houston, Snow Mountain, etc.). Alternatively, the isolated antibodies can be administered to subjects susceptible to Norovirus infection to confer passive or short-term immunity.

The invention will now be illustrated in greater detail by reference to the specific embodiments described in the following examples. The examples are intended to be purely illustrative of the invention and are not intended to limit its scope in any way.

### EXAMPLES

### Example 1. Dose Escalation, Safety and Immunogenicity Study of Intramuscular Norovirus Bivalent Virus-Like-Particle (VLP) Vaccine in Pediatric Subjects

A clinical study was undertaken to determine a safe, effective dosing level of Norovirus VLP vaccine for infants or children. The study was a phase 2, randomized, placebo-controlled, double-blind safety and immunogenicity trial in children aged 6 weeks to <9 years. Cohorts tested individually included children aged 6 weeks to <6 months; children aged 6 months to <1 year; children aged 1 year to <4 years; and children aged 4 years to <9 years. The Norovirus vaccine composition comprises GI.1 VLPs and GI1.4 VLPs (GI.1/GII.4c bivalent VLP vaccine). Four different dose levels were tested. Two of the dosing levels include the GI.1 and the GII.4 vaccine components at the same dosing level; two of the dosing levels include the GI.1 and the GII.4 vaccine components at two different dosing levels. The doses tested were:
GI.1/GII.4c VLP ratios of 15/15 μg,
GI.1/GII.4c VLP ratios of 15/50 μg,
GI.1/GII.4c VLP ratios of 50/50 and
GI.1/GII.4c VLP ratios of 50/150 μg.

The vaccine composition also included 500 μg Al(OH)₃ (adjuvant). The vaccine composition was administered to the subjects via intramuscular injection.

Two different dosing regimens were tested in the individual patient populations, as follows:
one dose or two doses in subjects aged 6 months to <9 years (i.e., in 6 months to <1 year group, the 1 year to <4 years group, and the 4 years to <9 years group); and
two or three doses in subjects aged 6 weeks to <6 months).

Inclusion Criteria included the following:
Male and female participants aged between 6 weeks and less than 9 years at the time of enrollment.
Are in good health at the time of entry into the trial as determined by medical history, physical examination (including vital signs) and clinical judgment of the investigator.
Participants legally authorized representative (LAR) signs and dates a written, informed consent form (ICF) and any required privacy authorization prior to the initiation of any trial procedures, after the nature of the trial has been explained according to local regulatory requirements. An assent will also be obtained according to age-appropriate country-specific regulations.
Participants who can comply with trial procedures and are available for the duration of the trial.

Exclusion Criteria included the following:
Clinically significant active infection (as assessed by the investigator) or body temperature 38.0° C. (100.4° F.) or higher within 3 days of the intended date of vaccination.
Have received antipyretic/analgesic medications within 24 hours prior to the intended vaccine administration.
Known hypersensitivity or allergy to investigational vaccine (including excipients of the investigational vaccines).
Behavioral or cognitive impairment or psychiatric disease that, in the opinion of the investigator, may interfere with the ability to participate in the trial.
History of any progressive or severe neurologic disorder, seizure disorder, or neuroinflammatory disease (eg, Guillain-Barre syndrome).
Known or suspected impairment/alteration of immune function, including the following:
Children <18 months of age with history of repeated episodes of acute otitis media (AOM) in the first 6 months of life (AOM defined as a bulging tympanic membrane) and not to be confused with otitis media with effusion (OME).
Chronic use of oral steroids (equivalent to 20 mg/day prednisone for ≥12 weeks/≥2 mg/kg body weight/day for ≥2 weeks) within 60 days prior to Day 1 (use of inhaled, intranasal, or topical corticosteroids is allowed).
Receipt of parenteral steroids (equivalent to 20 mg/day prednisone ≥12 weeks/≥2 mg/kg body weight/day for ≥2 weeks) within 60 days prior to Day 1.
Receipt of immunostimulants within 60 days prior to Day 1.
Receipt of parenteral, epidural, or intra-articular immunoglobulin preparation, blood products, and/or plasma derivatives within 3 months prior to Day 1 or planned during the full length of the trial.
Receipt of immunosuppressive therapy within 6 months prior to Day 1.
Human immunodeficiency virus (HIV) infection or HIV-related disease.
Chronic Hepatitis B or C infection.
Heritable immunodeficiency.
Abnormalities of splenic or thymic function.
Known bleeding diathesis or any condition that may be associated with a prolonged bleeding time.
Any serious chronic or progressive disease according to judgment of the investigator (e.g., neoplasm, insulin dependent diabetes, cardiac, renal, or hepatic disease).
Participation in any clinical trial with another investigational product 30 days prior to first trial visit or intent to participate in another clinical trial at any time during the conduct of this trial.
Received any other vaccines within 14 days (for inactivated vaccines) or 28 days (for live vaccines) prior to enrollment in this trial.
First degree relatives of individuals involved in trial conduct.
History of autoimmune disease.

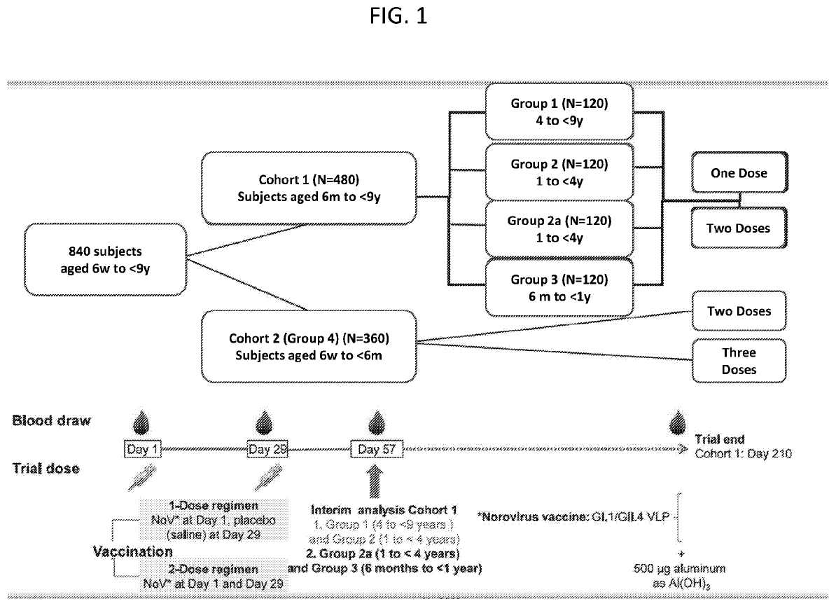

A schematic of the clinical study design is provided in FIG. 1. Cohort 1 included subjects in Groups 1 (4 years to <9 years of age), 2 (1 year to <4 years of age), 2a (1 year to <4 years of age), and 3 (6 months to <1 year of age). Group 2a is a bridging group allowing evaluation of two different manufacturing lots, prior to administration in the next younger age group. All 480 subjects in Cohort 1 received either 1 or 2 doses of GI.1/GII.4 VLP vaccine, at one of the dosing levels specified above (15/15, 15/50, 50/50, or 50/150). Cohort 2 included subjects of Group 4, aged 6 weeks to <6 months. All 360 subjects in Cohort 2 (Group 4) received two or three doses of GI.1/GII.4 VLP vaccine, at one of the dosing levels specified above (15/15, 15/50, 50/50, 50/150). Doses were administered at Day 1 (1-dose regimen), Days 1 and 29 (2-dose regimen, Cohort 1), Days 1 and 56 (2-dose regimen, Cohort 2), or Days 1, 56, and 112 (3-dose regimen, Cohort 2). Subjects in the 1 dose regimen received a placebo injection on Day 29 and subjects in the two-dose, Cohort 2 regimen received placebo injection on Day 112.

Table 1 below provides the demographics in the per-protocol patient set.

TABLE 1
Demographics

| | Group 1 | | Group 2 | | Group 2a | | Group 3 | | Group 4 | |
|---|---|---|---|---|---|---|---|---|---|---|
| Study Location | Finland, Panama, | | | | Panama, Colombia | | | | | |
| | Colombia | | | | | | | | | |
| N | 108 | | 112 | | 108 | | 108 | | 306 | |
| Age, mean (SD) | 5.8 yr | (1.39) | 2.1 yr | (0.85) | 2.0 yr | (0.74) | 8.1 mo | (1.44) | 3.1 mo | (1.32) |
| Gender, male, no. (%) | 52 | (48.1) | 60 | (53.6) | 61 | (56.5) | 57 | (52.8) | 157 | (51.3) |
| Race, no. (%) | | | | | | | | | | |
| White | 58 | (53.7) | 55 | (49.1) | 2 | (1.9) | 2 | (1.9) | 4 | (1.3) |
| American Indian or | 37 | (34.3) | 18 | (16.1) | 21 | (19.4) | 53 | (49.1) | 144 | (47.1) |
| Alaska Native | | | | | | | | | | |
| Black or African | 3 | (2.8) | 0 | | 18 | (16.7) | 12 | (11.1) | 40 | (13.1) |
| American | | | | | | | | | | |
| Other | 10 | (9.3) | 47 | (33.0) | 67 | (62.0) | 39 | (36.1) | 118 | (38.6) |

| | | | | | |
|---|---|---|---|---|---|
| Seronegative at | | | | | |
| baseline (%) | | | | | |
| GI.1-specific HBGA | 84 | 94 | 79 | 92 | 88 |
| GII.4c-specific HBGA | 30 | 55 | 44 | 82 | 58 |

The results of the study showed that all dosing levels were immunogenic. The 2-dose regimen of 50/150 μg dosing effective as well as safe in children aged 6 months to <5 years. The 3-dose regimen was effective as well as safe in children aged 6 weeks to <6 months.

### Immunogenicity

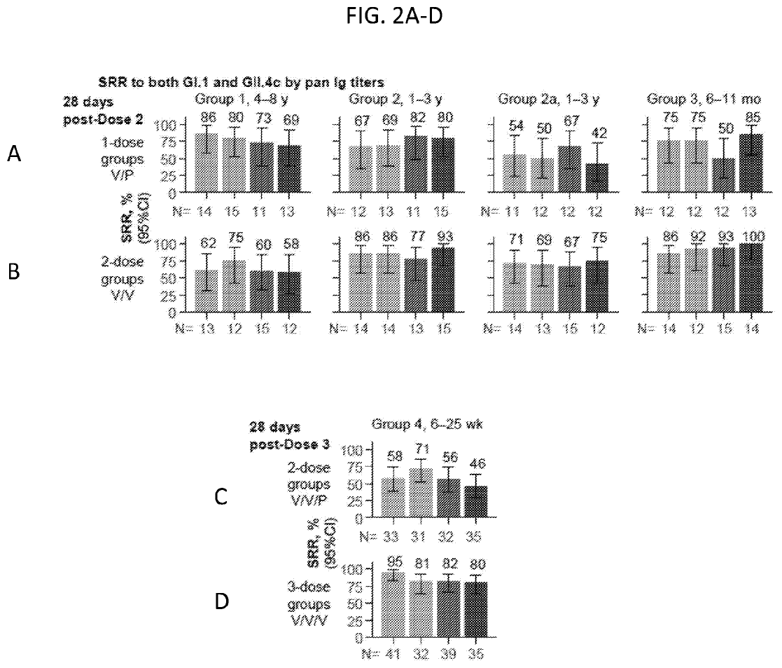

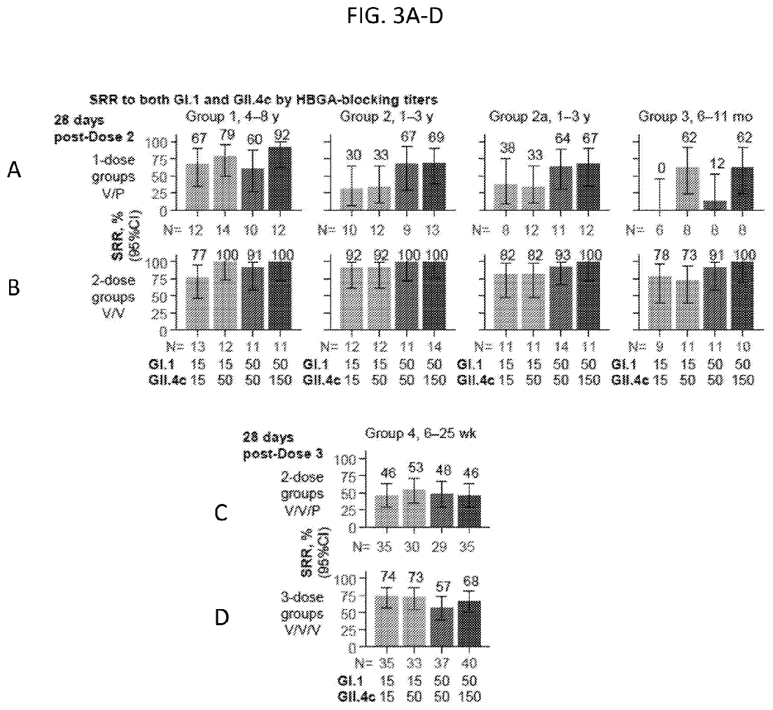

All dosing levels of the composition were immunogenic in the different age groups of children aged 6 weeks to <6 months, 6 to <12 months, 1 to <4 years, and 4 to <9 years (FIG. 2A-D). Based on HBGA-blocking titers, 2-doses of the vaccine composition appeared more immunogenic than 1 dose in Cohort 1 children aged 6 months to <1 year and 1 to <4 years (FIG. 3A, 3B). Based on pan-Ig and HBGA-blocking titers, 3-doses of the appeared more immunogenic than 2 doses in Cohort 2 infants aged 6 weeks to <6 months.

The majority of patients in Group 3 were seronegative at baseline (as measured by HBGA GI.1, HBGA GI1.4, pan-Ig GI.1, and pan-IG GII.4). Thus, the vaccine was immunogenic in mostly unprimed children. Similar patterns of immune responses as measured by HGBA blocking antibodies and pan-Ig were observed in the children age 1 to <4 years, who received the two manufacturing lots (Group 2 and Group 2a; FIGS. 2A, 2B, 3A, 3B). The two-dose vaccine regimen was more immunogenic than a single dose regimen in both age groups (6 months to >12 months and 1 to <4 years).

Table 2 below shows the seroresponse (4-fold rise in titer compared to baseline) to both GI.1 and GII.4, in combined formulations and age groups, for subjects with a baseline titer of below the lower limit of quantification (LLoQ) for both VLPs in all assays.

TABLE 2
Combined formulations and age groups: Groups 1 and 2
seroresponse to both GI.1 and GII.4 in patients
with baseline titer <LLoQ for both VLPs

| | | | |
|---|---|---|---|
| Visit | Pan-Ig | HBGA | IgA |

1-Dose Groups (Saline at date 29); ( N = 6)

| | | | |
|---|---|---|---|
| Day 29 SRR | 5/6 (83%) | 2/5 (40%) | 2/5 (40% |
| Day 57 SRR | 6/6 (100%) | 2/5 (40%) | 0/6 (0%) |

2-Dose Groups (N = 8)

| | | | |
|---|---|---|---|
| Day 29 SRR | 8/8 (100%) | 4/8 (50%) | 8/8 (100%) |
| Day 57 SRR | 8/8 (100%) | 8/8 (100%) | 7/8 (88%) |

Like the data from Group 3, the data summarized in Table 2 shows that the vaccine was immunogenic in unprimed children. The presence of GI.1-specific and GII.4c-specific maternal antibodies was suggested in Group 4 (6 weeks to <6 months) by the relatively low prevalence of subjects with seronegative GI.1-specific or GII.4c-specific pan-Ig antibodies.

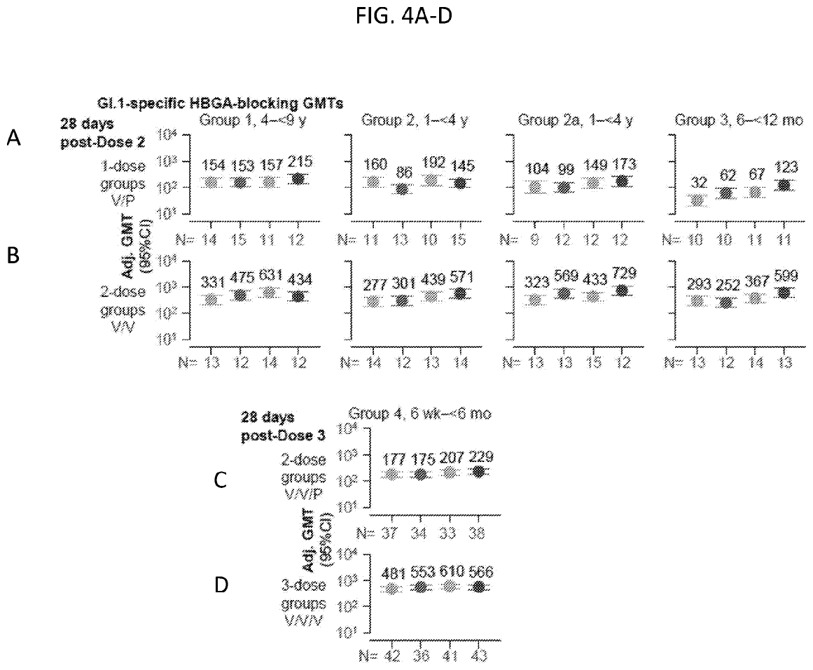

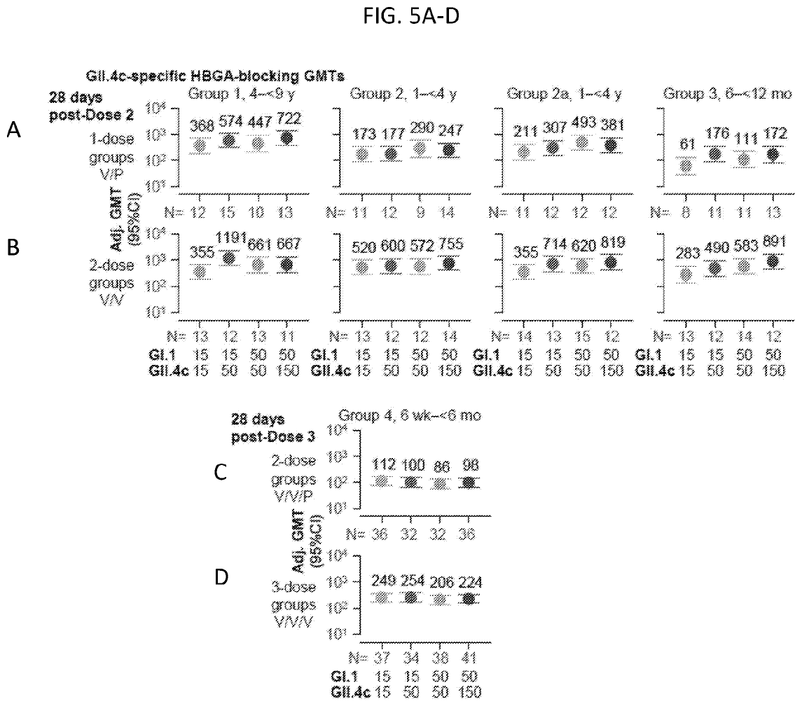

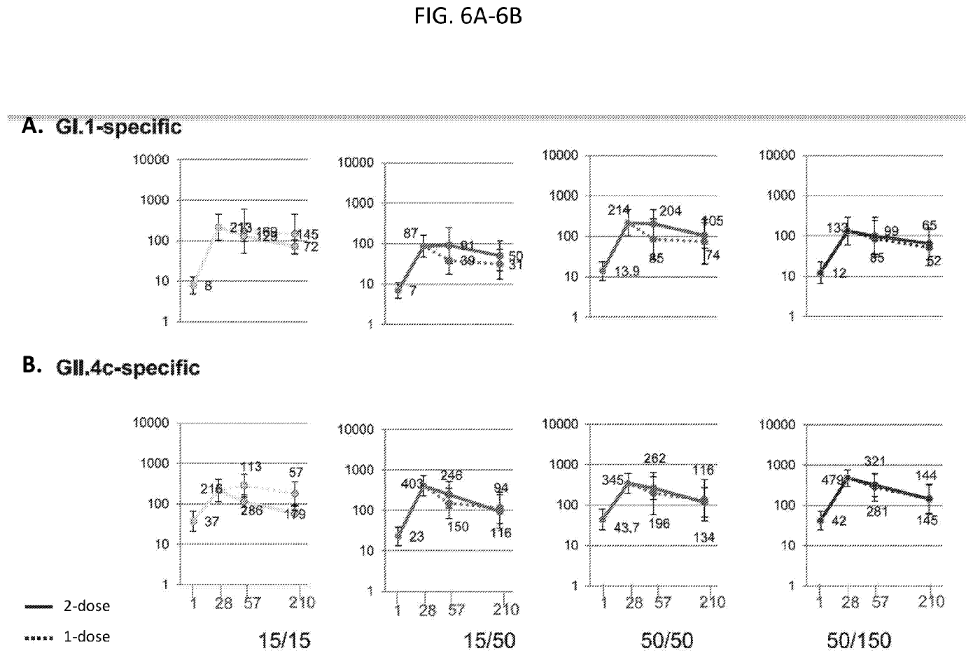

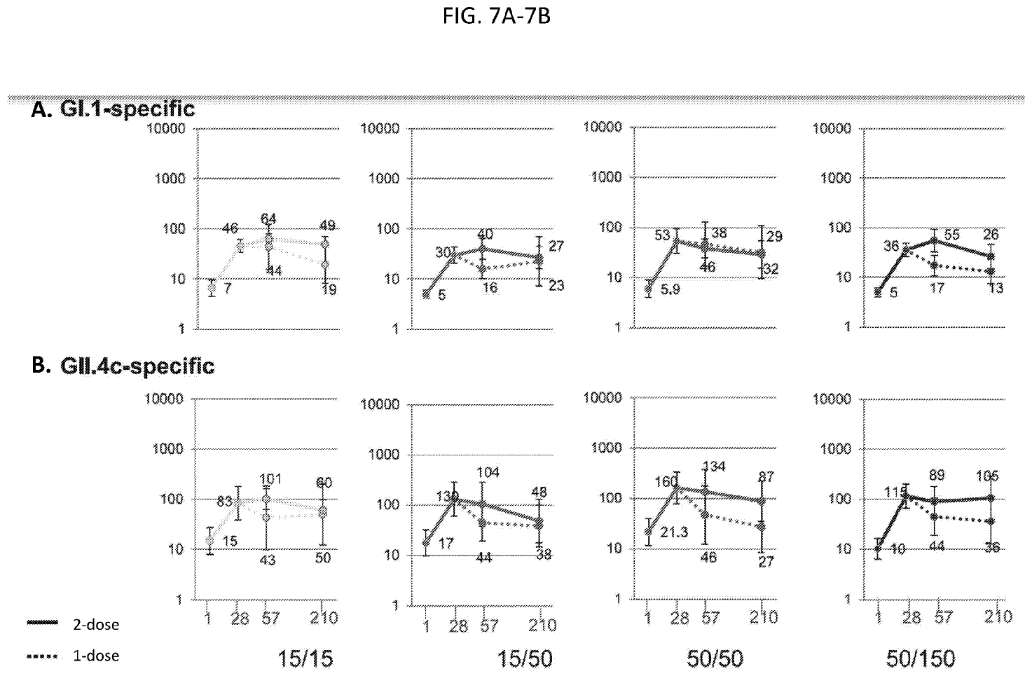

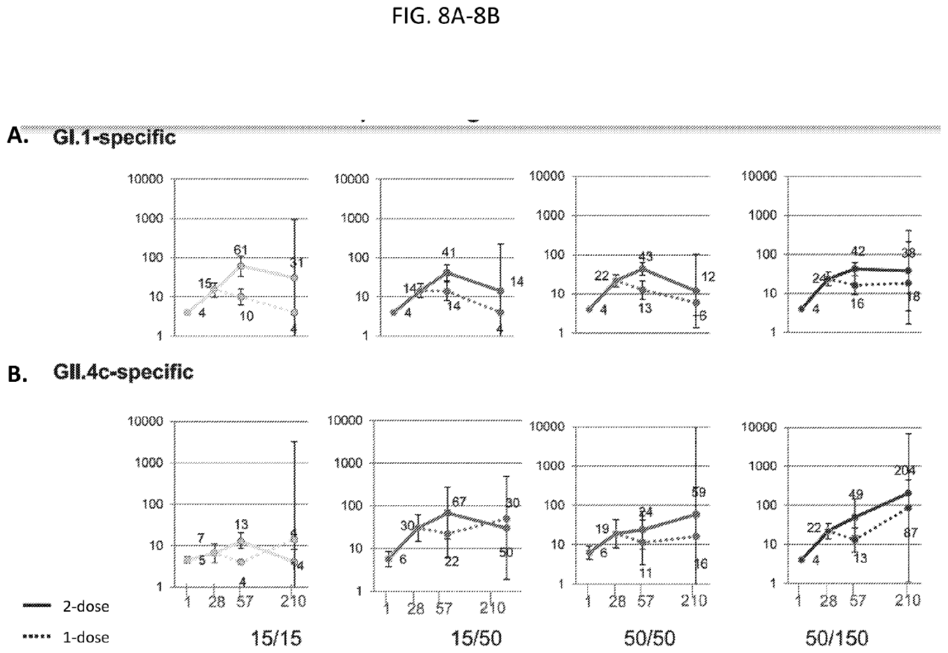

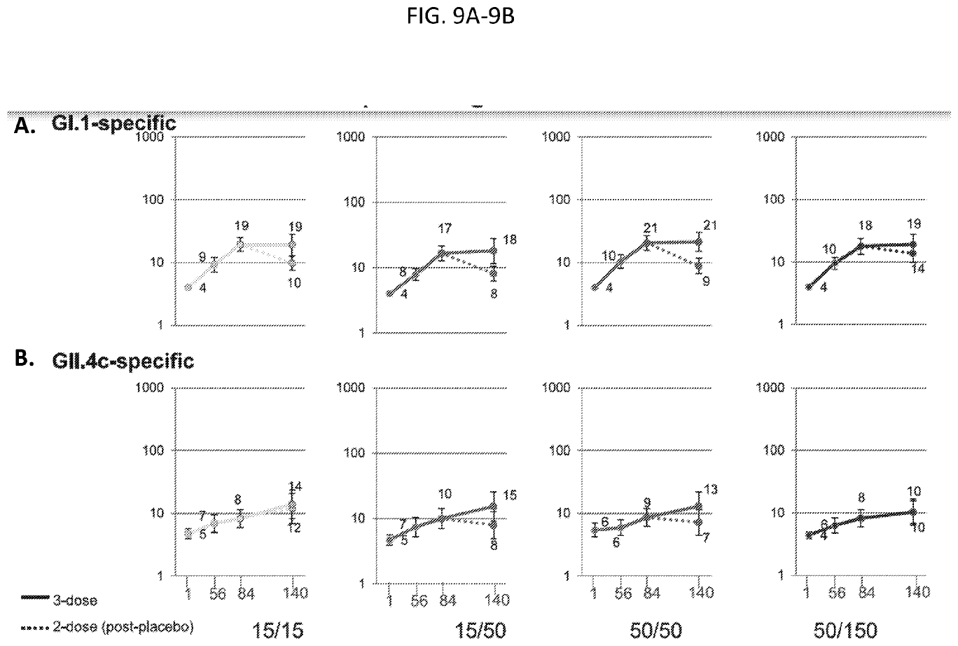

The 50/150 composition, in comparison with the other compositions, was associated with the highest GI.1-specific (FIGS. 4A-4D) and GII.4c-specific (FIG. 5A-5D) HBGA-blocking GMTs after 2 doses in Cohort 1 children aged 6 to <12 months, and aged 1 to <4 years, or after 3 doses in Cohort 2 infants aged 6 weeks to <6 months GMTs for GI.1-specific IGA and GII.4c-specific IgA are shown in FIGS. 6A-6B (Group 1), FIGS. 7A-7B (Group 2), FIGS. 8A-8B (Group 3), and FIGS. 9A-9B (Group 4).

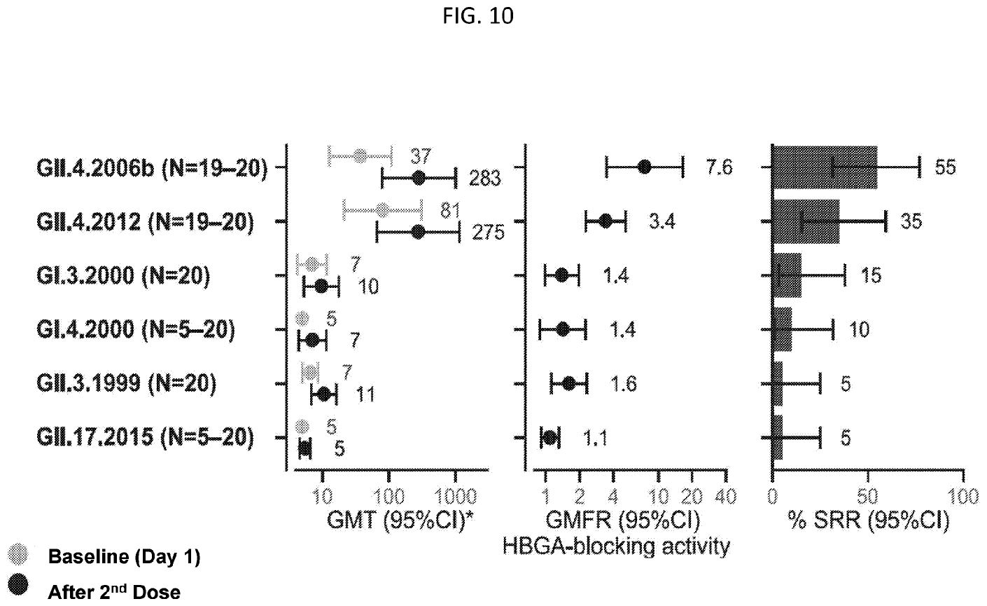

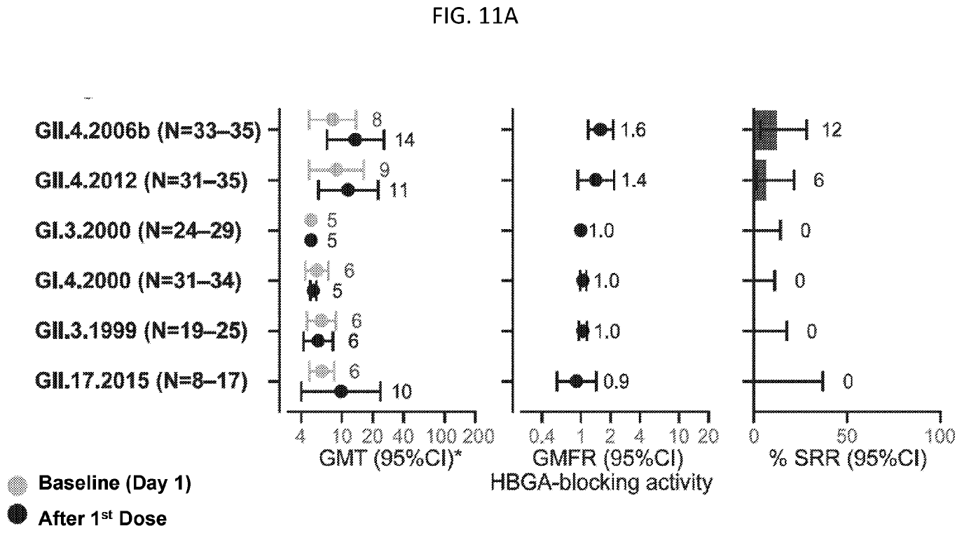

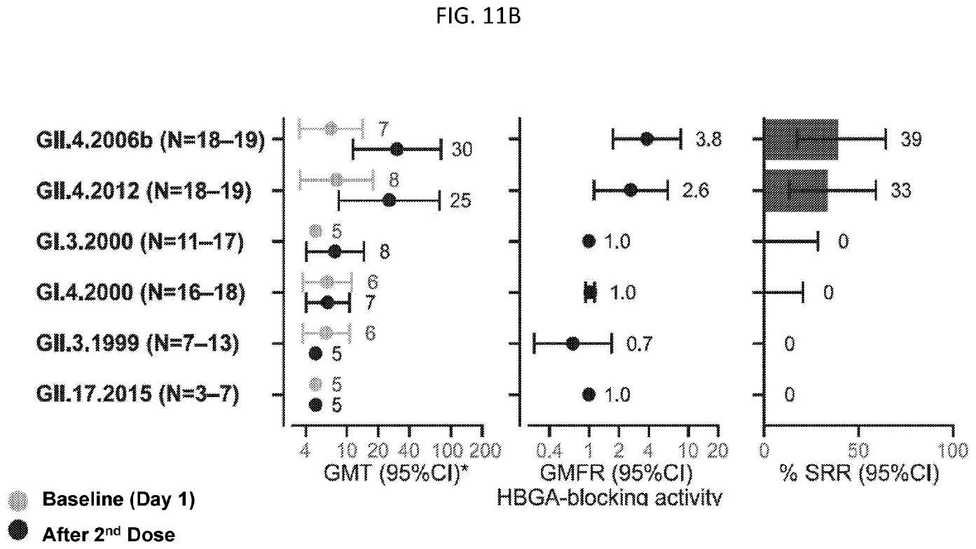

Cross-reactivity to other Norovirus strains was tested in Groups 2 and 3. In a subset of children aged 1 to <4 years (i.e., Group 2; composition groups combined), high cross-reactivity was observed with the heterovariant GII.4 VLPs (GII.4.2006b and GII.4.2012) after the second dose of vaccine (FIG. 10). In infants aged 6 to <12 months (i.e., Group 3, 2 dose group, composition groups combined), marginal cross-reactivity occurred after a single vaccine dose, only with the 2 heterovariant GII.4 strains GII.4.2006b and GII.4.2012 (FIG. 11A). After two vaccine doses, Group 3 subjects exhibited higher cross-reactivity with the 2 heterovariant GII.4 strains (FIG. 11B).

### Safety

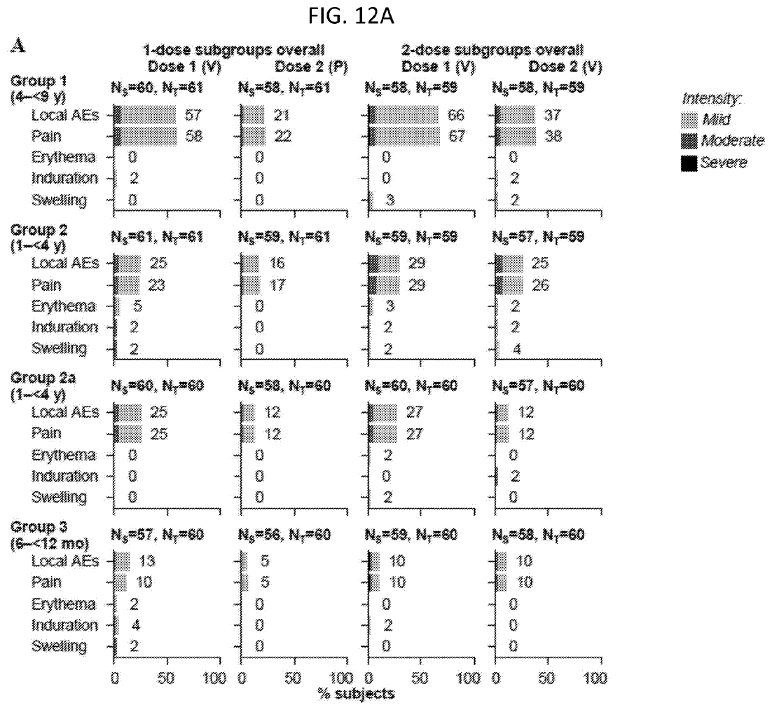

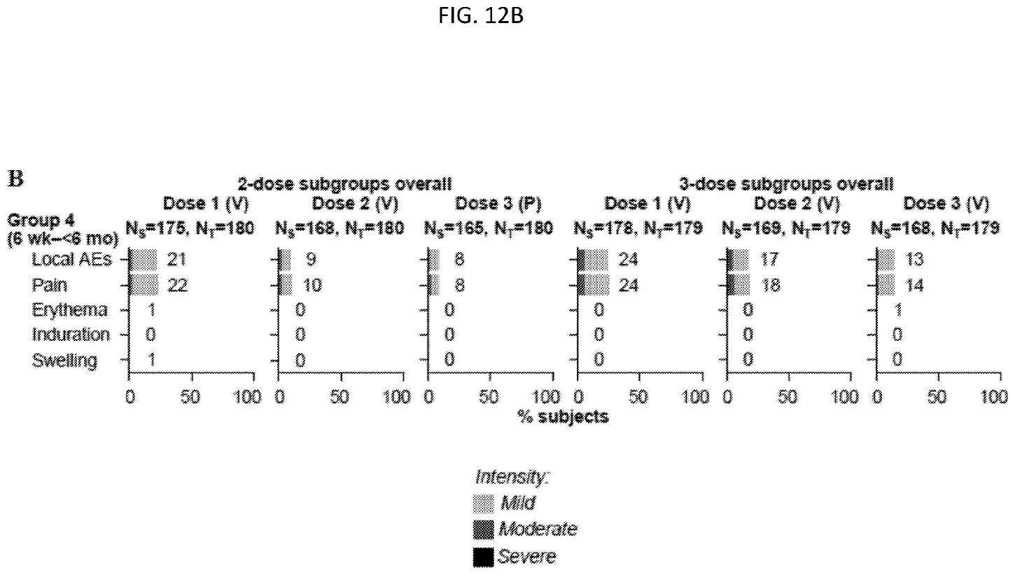

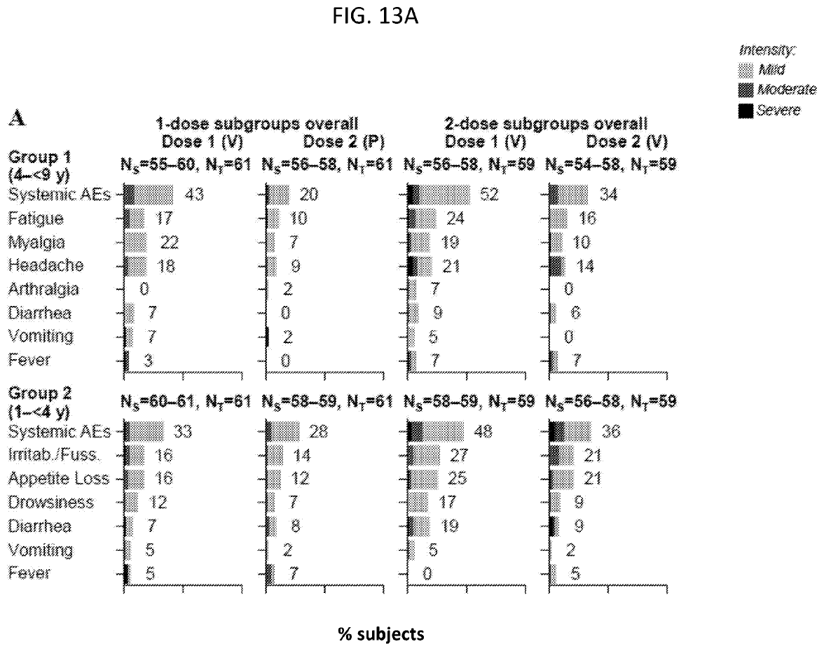

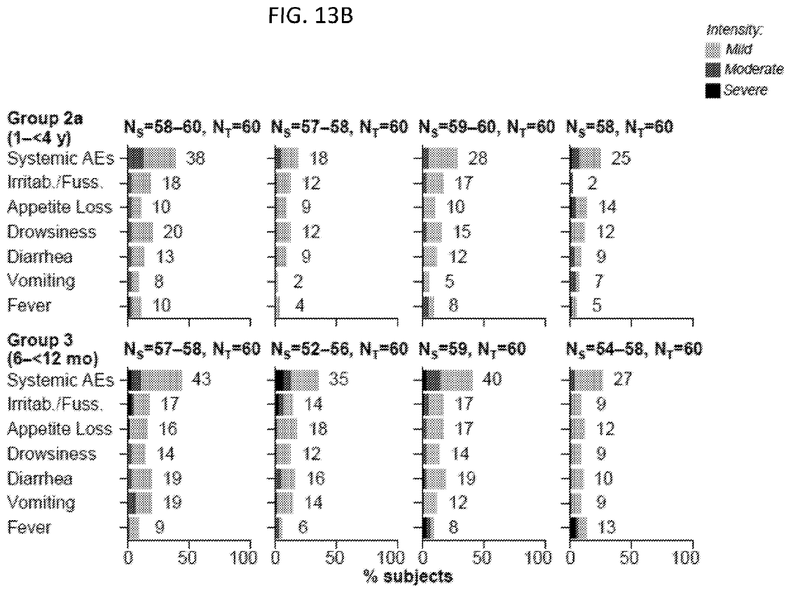

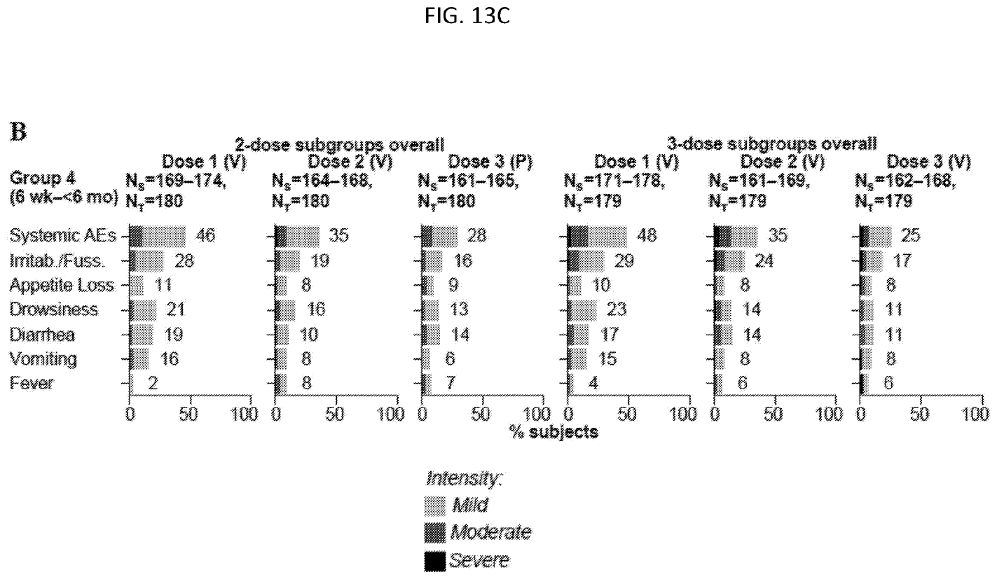

No vaccine-related severe adverse events (SAEs) or fatal SAEs were reported. The safety profiles for the different vaccine compositions (15/15, 15/50, 50/50, and 50/150) were similar for each pediatric age stratum and were primarily characterized by mild to moderate reactogenic symptoms, mostly mild to moderate in intensity, with severe symptoms being relatively infrequent (≤5% of subjects), of short duration, and with no increase after a second dose in either cohort or after the third dose in Cohort 2. The solicited local adverse events (AEs) for Groups 1, 2, 2A, and 3 are shown in FIG. 12A. The solicited local AEs for group 4 are shown in FIG. 12B. The solicited systemic AEs for Groups 1 and 2 are shown in FIG. 13A. The solicited systemic AEs for Groups 2a and 3 are shown in FIG. 13B. The solicited systemic AEs for Group 4 are shown in FIG. 13C.

Table 3 provides an overview of the unsolicited AEs, by arm and dose subgroup, after 28 days, across all Groups.

TABLE 3
Unsolicited AEs.

| | | Number of AEs (nₑ) & number (percentage of subjects; nₛ [%]) reporting the AE | | | | | | | | | | | |
|---|---|---|---|---|---|---|---|---|---|---|---|---|---|
| Dose^{(a)} | Sub- | A (15/15) | | | B (15/50) | | | C (50/50) | | | D (50/150) | | |
| (V or P) | group | N | nₑ | nₛ (%) | N | nₑ | nₛ (%) | N | nₑ | nₛ (%) | N | nₑ | nₛ (%) |

Group 1 (4-<9 y)

| | | | | | | | | | | | | | |
|---|---|---|---|---|---|---|---|---|---|---|---|---|---|
| 1 (V) | 1-Dose | 15 | 7 | 4 (27) | 16 | 5 | 5 (31) | 14 | 10 | 8 (57) | 16 | 11 | 6 (38) |
| 1 (V) | 2-Dose | 14 | 8 | 6 (43) | 14 | 11 | 5 (36) | 16 | 10 | 7 (44) | 15 | 6 | 5 (33) |
| 2 (P) | 1-Dose | 15 | 6 | 5 (33) | 16 | 9 | 5 (31) | 14 | 6 | 5 (36) | 16 | 6 | 5 (31) |
| 2 (V) | 2-Dose | 14 | 7 | 6 (43) | 14 | 4 | 2 (14) | 16 | 5 | 5 (31) | 15 | 5 | 5 (33) |

Group 2 (1-<4 y)

| | | | | | | | | | | | | | |
|---|---|---|---|---|---|---|---|---|---|---|---|---|---|
| 1 (V) | 1-Dose | 16 | 15 | 11 (69) | 16 | 14 | 10 (62) | 14 | 5 | 5 (36) | 15 | 6 | 6 (40) |
| 1 (V) | 2-Dose | 15 | 11 | 8 (53) | 14 | 10 | 6 (43) | 15 | 13 | 9 (60) | 15 | 11 | 8 (53) |
| 2 (P) | 1-Dose | 16 | 14 | 8 (50) | 16 | 10 | 7 (44) | 14 | 6 | 3 (21) | 15 | 4 | 4 (27) |
| 2 (V) | 2-Dose | 15 | 8 | 7 (47) | 14 | 12 | 6 (43) | 15 | 8 | 5 (33) | 15 | 8 | 8 (53) |

Group 2a (1-<4 y)

| | | | | | | | | | | | | | |
|---|---|---|---|---|---|---|---|---|---|---|---|---|---|
| 1 (V) | 1-Dose | 14 | 3 | 3 (21) | 15 | 9 | 7 (47) | 15 | 9 | 5 (33) | 16 | 7 | 5 (31) |
| 1 (V) | 2-Dose | 16 | 6 | 6 (38) | 15 | 7 | 6 (40) | 15 | 3 | 3 (20) | 14 | 2 | 2 (14) |
| 2 (P) | 1-Dose | 14 | 6 | 4 (29) | 15 | 7 | 5 (33) | 15 | 6 | 6 (40) | 16 | 5 | 5 (31) |
| 2 (V) | 2-Dose | 16 | 2 | 2 (12) | 15 | 6 | 6 (40) | 15 | 7 | 6 (40) | 14 | 7 | 6 (43) |

Group 3 (6-<12 mo)

| | | | | | | | | | | | | | |
|---|---|---|---|---|---|---|---|---|---|---|---|---|---|
| 1 (V) | 1-Dose | 16 | 10 | 8 (53) | 15 | 7 | 6 (40) | 15 | 7 | 5 (33) | 15 | 10 | 8 (53) |
| 1 (V) | 2-Dose | 15 | 10 | 8 (53) | 15 | 10 | 7 (47) | 15 | 7 | 6 (40) | 15 | 5 | 5 (33) |
| 2 (P) | 1-Dose | 15 | 10 | 8 (53) | 15 | 7 | 6 (40) | 15 | 12 | 9 (60) | 15 | 11 | 10 (67) |
| 2 (V) | 2-Dose | 15 | 13 | 8 (53) | 15 | 14 | 12 (80) | 15 | 7 | 6 (40) | 15 | 9 | 5 (33) |

Group 4 (6 wk-<6 mo)

| | | | | | | | | | | | | | |
|---|---|---|---|---|---|---|---|---|---|---|---|---|---|
| 1 (V) | 2-Dose | 45 | 34 | 24 (53) | 46 | 32 | 20 (44) | 44 | 31 | 23 (52) | 45 | 21 | 15 (33) |
| 1 (V) | 3-Dose | 45 | 27 | 18 (40) | 44 | 26 | 20 (46) | 46 | 22 | 19 (41) | 44 | 22 | 16 (36) |
| 2 (V) | 2-Dose | 45 | 25 | 18 (40) | 46 | 27 | 21 (46) | 44 | 33 | 26 (59) | 45 | 28 | 21 (47) |
| 2 (V) | 3-Dose | 45 | 24 | 19 (42) | 44 | 24 | 16 (36) | 46 | 30 | 24 (52) | 44 | 40 | 24 (54) |
| 3 (P) | 2-Dose | 45 | 21 | 17 (38) | 46 | 16 | 14 (30) | 44 | 25 | 19 (43) | 45 | 21 | 17 (38) |
| 3 (V) | 3-Dose | 45 | 21 | 14 (31) | 44 | 20 | 16 (36) | 46 | 33 | 22 (48) | 44 | 26 | 19 (43) |

Table 4 provides an overview of SAEs by arm and dose subgroup, for all groups.

TABLE 4
SAEs

| | Number of SAEs (nₑ) & number (percentage of subjects; nₛ [%]) reporting the SAE | | | | | | | | | | | |
|---|---|---|---|---|---|---|---|---|---|---|---|---|
| Group/Sub- | A (15/15) | | | B (15/50) | | | C (50/50) | | | D (50/150) | | |
| group^{(a)} | N | nₑ | nₛ (%) | N | nₑ | nₛ (%) | N | nₑ | nₛ (%) | N | nₑ | nₛ (%) |

Group 1 (4-<9 y)

| | | | | | | | | | | | | |
|---|---|---|---|---|---|---|---|---|---|---|---|---|
| 1-Dose | 15 | 0 | 0 (0) | 16 | 0 | 0 (0) | 14 | 0 | 0 (0) | 16 | 1 | 1 (6) |
| 2-Dose | 14 | 0 | 0 (0) | 14 | 0 | 0 (0) | 16 | 1 | 1 (6) | 15 | 1 | 1 (7) |

Group 2 (1-<4 y)

| | | | | | | | | | | | | |
|---|---|---|---|---|---|---|---|---|---|---|---|---|
| 1-Dose | 16 | 0 | 0 (0) | 16 | 0 | 0 (0) | 14 | 0 | 0 (0) | 15 | 1 | 1 (7) |
| 2-Dose | 15 | 0 | 0 (0) | 14 | 0 | 0 (0) | 15 | 1 | 1 (7) | 15 | 1 | 1 (7) |

Group 2a (1-<4 y)

| | | | | | | | | | | | | |
|---|---|---|---|---|---|---|---|---|---|---|---|---|
| 1-Dose | 14 | 0 | 0 (0) | 15 | 4 | 3 (20) | 15 | 2 | 2 (13) | 16 | 1 | 1 (6) |
| 2-Dose | 16 | 0 | 0 (0) | 15 | 1 | 1 (7) | 15 | 0 | 0 (0) | 14 | 0 | 0 (0) |

Group 3 (6-<12 mo)

| | | | | | | | | | | | | |
|---|---|---|---|---|---|---|---|---|---|---|---|---|
| 1-Dose | 15 | 2 | 2 (13) | 15 | 1 | 1 (7) | 15 | 3 | 3 (20) | 15 | 0 | 0 (0) |
| 2-Dose | 15 | 2 | 2 (13) | 15 | 2 | 2 (13) | 15 | 0 | 0 (0) | 15 | 1 | 1 (7) |

Group 4 (6 wk-<6 mo)

| | | | | | | | | | | | | |
|---|---|---|---|---|---|---|---|---|---|---|---|---|
| 2-Dose | 45 | 5 | 5 (11) | 46 | 4 | 4 (9) | 44 | 5 | 4 (9) | 45 | 4 | 4 (9) |
| 3-Dose | 45 | 12 | 11 (24) | 44 | 6 | 4 (9) | 46 | 4 | 3 (6) | 44 | 7 | 6 (14) |

The data from the study revealed that the bivalent Norovirus vaccine composition was safe even at the highest doses and highest numbers of doses administered in the study. There was no apparent association between the frequencies of solicited AEs and antigen quantity in the vaccine. There was no increase in frequency of solicited AEs after the 2^{nd} dose, or after the 3^{rd} dose in Group 4. After the first vaccine dose, across the groups solicited AEs were reported for 39 to 57% of subjects. After the second vaccine dose, across the groups solicited AEs were reported for 37 to 46% of subjects. After dose 3, the frequencies of solicited AEs were similar for placebo and vaccine groups. After any vaccine dose, pain was the most frequent local system (≤24%); irritability (≤29%) and drowsiness (≤23%) were frequent systemic symptoms. Solicited symptoms were mostly mild or moderate in intensity. Similarly, most unsolicited AEs were of mild to moderate intensity, and were unrelated to the vaccine. None of the 38 SAEs reported for 24 subjects were related to the vaccine. No deaths were reported, and no AE led to withdrawal from the protocol.

Taken together, the study showed that the 50/150 μg composition of the GI.1/GII.4c bivalent vaccine, further comprising 500 μg aluminum as Al(OH)₃, was highly immunogenic and safe in children aged 6 weeks to <9 years. No safety concerns were identified, and therefore the composition has an acceptable safety profile in this population of patients, even in the context of 2 or 3 doses. Based on the safety and immunogenicity data obtained via the clinical study, a pediatric population of patients can be dosed with 2 or 3 doses of a composition comprising 50 μg GI.1 VLP and 150 μg GII.4 VLP. For example, a population of patients aged 6 weeks to <6 months can safely and effectively be administered 3 doses of the composition. A population of patients aged 6 months to <9 years can safely and effectively be administered 2 doses of the composition.

Thus, in some embodiments, the present disclosure provides methods, uses and compostions for use in inducing protective immunity against Norovirus infection comprising first determining a subject's age, and then determining the number of doses to be administered to the subject. For example, if the subject's age is at least about 6 weeks but less than 6 months, then the subject is administered a Norovirus VLP vaccine (e.g., GI.1/GII.4c bivalent vaccine at a dosing level of 50 μg/150 μg) in three separate doses; and if the subject's age is at least about 6 months but less than 9 years, then the subject is administered a Norovirus VLP vaccine (e.g., GI.1/GII.4c bivalent vaccine at a dosing level of 50 μg/150 μg) in two separate doses.

The present invention is not to be limited in scope by the specific embodiments described which are intended as single illustrations of individual aspects of the invention, and functionally equivalent methods and components are within the scope of the invention. Indeed, various modifications of the invention, in addition to those shown and described herein, will become apparent to those skilled in the art from the foregoing description and accompanying drawings using no more than routine experimentation. Such modifications and equivalents are intended to fall within the scope of the appended claims.

All publications, patents and patent applications mentioned in this specification are herein incorporated by reference into the specification to the same extent as if each individual publication, patent or patent application was specifically and individually indicated to be incorporated herein by reference.

Citation or discussion of a reference herein shall not be construed as an admission that such is prior art to the present invention.

1. A method for inducing protective immunity against Norovirus infection in a pediatric subject, comprising administering to the subject a composition comprising Norovirus virus-like particles (VLPs) in a dosing regimen, wherein the dosing regimen comprises administering the composition in at least a first dose and a second dose.

2. The method of claim 1, wherein the composition comprises Norovirus genogroup I VLPs and Norovirus genogroup II VLPs.

3. The method of claim 1 or claim 2, wherein the composition comprises Norovirus genogroup I, genotype 1 (GI.1) VLPs and Norovirus genogroup II, genotype 4 (GII.4) VLPs.

4. The method of claim 3, wherein the GII.4 VLPs are derived from expression of a consensus sequence of circulating strains of Norovirus genogroup II, genotype 4.

5. The method of claim 4, wherein the GII.4 VLPs comprise a capsid protein comprising a sequence of SEQ ID NO: 1.

6. The method of any one of claims 1-5, wherein the composition comprises about 50 μg Norovirus genogroup I VLPs and about 150 μg Norovirus genogroup II VLPs.

7. The method of any one of claims 1-7, wherein the pediatric subject is between about 6 weeks and about 9 years of age.

8. The method of any one of claims 1-7, wherein the pediatric subject is between about 6 weeks and about 6 months of age.

9. The method of claim 8, wherein the composition is administered to the subject in three doses.

10. The method of any one of claims 1-7, wherein the pediatric subject is between about 6 months and about 9 years of age.

11. The method of claim 9, wherein the composition is administered to the subject in no more than two doses.

12. The method of any one of claims 1-7, wherein the first and second doses are administered to the subject about 1, about 2, or about 3 months apart.

13. The method of any one of claims 1-7, the first dose is administered when the subject about 5 months of age and the second dose is administered when the subject is about 7 months of age.

14. The method of any one of claims 1-13, wherein the composition is administered to the subject intramuscularly.

15. The method of any one of claims 1-14, wherein the composition comprises at least one adjuvant.

16. The method of any one of claims 1-14, wherein the composition comprises a single adjuvant.

17. The method of claim 15 or 16, wherein the adjuvant is aluminum hydroxide.

18. The method of any one of claims 1-17, wherein the composition comprises 500 μg of aluminum hydroxide.

19. The method of any one of claims 1-18, wherein the method elicits at least a three-fold increase in Norovirus-specific serum antibody titer as compared to the titer in the subject prior to administration of the composition.

20. The method of any one of claims 1-19, wherein the method is associated with an acceptable safety profile and/or wherein the administration of the composition is well tolerated in the pediatric subject.

21. The method of any one of claims 1-20, wherein the method induces cross-reactivity to one or more viral strains not present in the composition.

22. A composition comprising genogroup I Norovirus VLPs and genogroup II Norovirus VLPs for use in a method of eliciting protective immunity against norovirus in a pediatric subject.

23. The composition for use according to claim 22, wherein the Norovirus genogroup I VLPs are genogroup I, genotype 1 (GI.1) VLPs and wherein the Norovirus genogroup II VLPs are Norovirus genogroup II, genotype 4 (GII.4) VLPs.

24. The composition for use according to claim 23, wherein the GII.4 VLPs are derived from expression of a consensus sequence of circulating strains of Norovirus genogroup II, genotype 4.

25. The composition for use according to claim 24, wherein the GII.4 VLPs comprise a capsid protein comprising a sequence of SEQ ID NO: 1.

26. The composition for use according to any one of claims 22-25, wherein the composition comprises about 50 μg Norovirus genogroup I VLPs and about 150 μg Norovirus genogroup II VLPs.

27. The composition for use according to any one of claims 22-26, wherein the pediatric subject is between about 6 weeks and about 9 years of age.

28. The composition for use according to any one of claims 22-27, wherein the pediatric subject is between about 6 weeks and about 6 months of age.

29. The composition for use according to claim 28, wherein the composition is administered to the subject in three doses.

30. The composition for use according to any one of claims 22-27, wherein the pediatric subject is between about 6 months and about 9 years of age.

31. The composition for use according to claim 30, wherein the composition is administered to the subject in no more than two doses.

32. The composition for use according to any one of claims 22-27, wherein the composition is administered to the subject in at least a first dose and a second dose, wherein the first and second doses are administered to the subject about 1, about 2, or about 3 months apart.

33. The composition for use according to claim 32, wherein the first dose is administered when the subject about 5 months of age and the second dose is administered when the subject is about 7 months of age.

34. The composition for use according to any one of claims 22-33, wherein the composition is administered to the subject intramuscularly.

35. The composition for use according to any one of claims 22-34, wherein the composition comprises at least one adjuvant.

36. The composition for use according to any one of claims 22-34, wherein the composition comprises a single adjuvant.

37. The composition for use according to claim 35 or 36, wherein the adjuvant is aluminum hydroxide.

38. The composition for use according to any one of claims 22-34, wherein the composition comprises 500 μg of aluminum hydroxide.

39. The composition for use according to any one of claims 22-38, wherein the composition induces cross-reactivity to one or more viral strains not present in the composition.

40. Use of a composition comprising genogroup I Norovirus VLPs and genogroup II Norovirus VLPs in a method for eliciting protective immunity against Norovirus infection in a pediatric subject.

41. The use of claim 40, wherein the Norovirus genogroup I VLPs are genogroup I, genotype 1 (GI.1) VLPs and wherein the Norovirus genogroup II VLPs are Norovirus genogroup II, genotype 4 (GII.4) VLPs.

42. The use of claim 41, wherein the GII.4 VLPs are derived from expression of a consensus sequence of circulating strains of Norovirus genogroup II, genotype 4.

43. The use of claim 42, wherein the GII.4 VLPs comprise a capsid protein comprising a sequence of SEQ ID NO: 1.

44. The use according to any one of claims 40-43, wherein the composition comprises about 50 μg Norovirus genogroup I VLPs and about 150 μg Norovirus genogroup II VLPs.

45. The use according to any one of claims 40-44, wherein the pediatric subject is between about 6 weeks and about 9 years of age.

46. The use according to any one of claims 40-45, wherein the pediatric subject is between about 6 weeks and about 6 months of age.

47. The use according to claim 46, wherein the composition is administered to the subject in three doses.

48. The use according to any one of claims 40-45, wherein the pediatric subject is between about 6 months and about 9 years of age.

49. The use according to claim 48, wherein the composition is administered to the subject in no more than two doses.

50. The use according to any one of claims 40-45, wherein the composition is administered to the subject in at least a first dose and a second dose, wherein the first and second doses are administered to the subject about 1, about 2, or about 3 months apart.

51. The use according to claim 50, wherein the first dose is administered when the subject about 5 months of age and the second dose is administered when the subject is about 7 months of age.

52. The use according to any one of claims 40-51, wherein the composition is administered to the subject intramuscularly.

53. The use according to any one of claims 40-52, wherein the composition comprises at least one adjuvant.

54. The use according to any one of claims 40-52, wherein the composition comprises a single adjuvant.

55. The use according to claim 53 or 54, wherein the adjuvant is aluminum hydroxide.

56. The use according to any one of claims 40-52, wherein the composition comprises 500 μg of aluminum hydroxide.

57. The use according to any one of claims 40-52, wherein the composition induces cross-reactivity against one or more viral strains not present in the composition.

58. A method for inducing protective immunity against Norovirus infection in a pediatric subject, comprising the steps of determining the subject's age; and
(ii) (a) if the age is at least about 6 weeks but less than 6 months, administering to the subject a composition comprising Norovirus virus-like particles (VLPs) in a dosing regimen consisting of administering the composition in three separate doses, and
(b) if the age is at least 6 months but less than 9 years, administering to the subject a composition comprising Norovirus virus-like particles (VLPs) in a dosing regimen consisting of administering the composition in two separate doses.
